(19) United States
(12) Patent Application Publication
(10) Pub. No.: US 2025/0163015 A1
(43) Pub. Date: May 22, 2025

(54) DITHIOLATE-BASED COMPOUNDS

(71) Applicant: The Government of the United States of America, as represented by the Secretary of the Navy, Arlington, VA (US)

(72) Inventors: Eunkeu Oh, Alexandria, VA (US); Kimihiro Susumu, Alexandria, VA (US); Scott A. Walper, Richmond (GB); Jozsef Czege, Highland, MD (US)

(73) Assignee: The Government of the United States of America, as represented by the Secretary of the Navy, Arlington, VA (US)

(21) Appl. No.: 18/952,217

(22) Filed: Nov. 19, 2024

Related U.S. Application Data
(60) Provisional application No. 63/601,560, filed on Nov. 21, 2023.

(51) Int. Cl. C07D 339/04; C07C 321/04

(52) U.S. Cl. CPC C07D 339/04; C07C 321/04

(57) ABSTRACT

The compounds shown below are presented. The compounds have a bidentate binding group: thioctic acid (TA) with disulfide or its reduced form of dihydrolipoic acid (DHLA) with dithiol. In embodiments, the compounds form part of a dithiolate-based ligand-grafted gold nanoparticle (AuNP). A method includes: mixing a testing material with a dithiolate-based ligand-grafted AuNP probe in solution, thereby generating a test sample; chilling the test sample at a predetermined temperature for a period of time; subsequent to chilling the test sample, detecting a color of the test sample; and determining anti-icing effects of the testing material based on the color of the test sample.

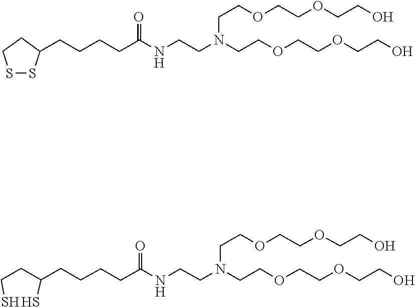 [SMILES: O=C(CCCCC1CCSS1)NCCN(CCOCCOCCO)CCOCCOCCO ; O=C(CCCCC(S)CCS)NCCN(CCOCCOCCO)CCOCCOCCO]

### CROSS-REFERENCE TO RELATED APPLICATIONS

This application is a Nonprovisional of, and claims the benefit of priority under 35 U.S.C. § 119 based on, U.S. Provisional Patent Application No. 63/601,560 filed Nov. 21, 2023. The Provisional application and all references cited herein are hereby incorporated by reference into the present disclosure in their entirety.

### FEDERALLY SPONSORED RESEARCH AND DEVELOPMENT

The United States Government has ownership rights in this invention. Licensing inquiries may be directed to Office of Technology Transfer, US Naval Research Laboratory, Code 1004, Washington, D.C. 20375, USA; +1.202.767.7230; nrltechtran@us.navy.mil, referencing Navy Case No. 211784-US3.

### BACKGROUND OF THE INVENTION

Aspects of the present invention relate generally to colorimetric assays and, more particularly, to colorimetric assays using dithiolate-grafted nanoparticles (NPs) for high-throughput screening (HTS) of cryoprotectants or anti-icing materials.

Gold nanoparticles (AuNP) in solution have been previously utilized in colorimetric assays. AuNPs have a unique surface plasmon resonance (SPR) peak near 520 nanometers (nm) (for 10-20 nm diameter nanoparticles) when colloidal stability is intact and AuNPs are well dispersed in solution. However, AuNPs in solution have been shown to irreversibly aggregate or cluster together during the freezing process. This results in significant changes to the detected spectrum and observed color of the AuNP solution after freezing and thawing of the solution.

Anti-icing studies have been dramatically increasing for the last decade due to the increasing demands of cryopreservation, organ banking, and sustainability in cold environments. Most of the studies have focused on developing new anti-icing materials such as chemical-based cryoprotectants, or biological molecule-based cryoprotectants such as peptides and antifreeze proteins (AFPs), antifreeze glycol proteins (AFGPs), or ice binding proteins (IBPs). AFPs and IBPs were first isolated from the blood serum of polar fishes, but various types of similar proteins have been found from numerous origins (e.g., fungus, plant, fish, and bug). Some AFPs have shown specific interactions with ice or unique binding affinities to certain facets of ice, and also to cellular membranes, to prolong the life time of organisms (over 0° C.).

The most common aspects to look for when testing or analyzing materials for anti-icing activity are: (1) thermal hysteresis (TH); (2) ice recrystallization inhibition (IRI); and (3) dynamic ice shaping (DIS). In general, TH is the ability of a test material to lower the freezing point of aqueous solutions relative to the melting point. As used herein, IRI refers to the suppression, by a test material, of ice crystal regrowth of frozen samples during temperature changes. Assays utilized to determine IRI include “splat assays” to analyze the granule size of ice crystals of a super-thin sample, and “sandwich assays” to monitor the ice crystal size in a super-cooled solution using high concentration of sucrose or other types of carbohydrates. As used herein, DIS refers to the ability of a material (e.g., anti-icing material) to influence the morphology of a single ice crystal.

In order to develop new materials that contain anti-icing activity, there is a need for a high-throughput analysis method for the fast screening of target materials or molecules from various sources. However, the traditional techniques described above (e.g., TH, IRI or DIS) require difficult sample preparation and very time-consuming observations (e.g., several hours to days) of slow ice growth under a microscope specially equipped with a cooling stage (e.g., a CryoStage with liquid nitrogen purging) having a high precision (e.g., <0.1° C.) of temperature control. Also, these microscope-based experiments can be performed only for a single condition (e.g., the specific concentration, fixed buffer, or reagents condition) and need to be compared to strict controls at the time of the experiment, which is not easily set up for unknown samples with different levels of anti-icing efficacy. Finally, the resulting imaging data require a significant amount of time and effort for image processing. The above-identified techniques thus demand a high level of technical skill, but have a low robustness with a high dependency on specific experimental conditions. Accordingly, it is very difficult to repeat or to compare the results of such techniques with different conditions or target materials. The nature of these types of traditional assays provide significant obstacles to their use in high-speed tests with standardization of products, and for their use in the HTS of new test materials.

One previously proposed HTS method comprised a colorimetric assay using gold nanoparticles (AuNPs) for the screening of anti-icing activity. AuNPs aggregated during the freezing process when no blocking material (anti-icing material) was present, which resulted in a color change within test samples. Using this method, absorption changes of AuNP were used to track color changes after freezing a sample using single thiol-grafted AuNPs (mercaptosuccinic acid (MSA)). Reported results demonstrated the fast speed of detection compared to a traditional TH assay.

In general, AuNPs have a unique surface plasmon resonance (SPR) peak near 520 nanometers (nm) (for 10˜20 nm diameter AuNPs) when the colloidal stability is intact and AuNPs are well dispersed in a solution. However, an SPR peak shifts to a longer wavelength or decreases the absorption at an original SPR peak position when AuNPs have some interaction with a chemical on the surface of AuNP aggregates after a freezing process (1˜2 hour of freezing process in −20° C.). Accordingly, the previously proposed AuNP method measured the absorption of AuNP in liquid at the moment of post-thaw and compared the amount of absorption changes due to the irreversible aggregation of AuNP-MSA (AuNP coated with MSA) with that of a control sample to measure the anti-icing effect (IRI or TH) of reagents. However, this method illustrated significant limitations including: (1) AuNP-MSA demonstrated low colloidal stability due to a single-thiolate ligand that caused false signals of aggregation in high ionic conditions (1×PBS, Tris/Borate, EDTA, or acetate buffer) or different pH conditions (beyond neutral, pH˜7) even before a freezing test; (2) spectrum measurements at a liquid phase of the post-thaw sample (when AuNP was already warmed up and loosely aggregated or dispersed) can limit the information of “freezing” that can bring direct information about anti-icing activity during ice-recrystallization or freezing suppression; and (3) spectrum comparisons are time consuming and are not a standardized test method without a control sample, such that it is not easy to compare results to those of another experiment or another batch of the same experiment.

Based on the above, there remains a need for a high-throughput analysis method for the fast screening of target materials for anti-icing activity.

### SUMMARY OF THE INVENTION

In a first aspect of the invention, there is a method including mixing a testing material with a dithiolate-based ligand-grafted gold nanoparticle (AuNP) probe in solution, thereby generating a test sample; chilling the test sample at a predetermined temperature for a period of time; subsequent to chilling the test sample, detecting a color of the test sample; and determining anti-icing effects of the testing material based on the color of the test sample. In aspects, the temperature is a temperature below freezing, and the time period is a period long enough to bring the temperature of the test sample to a temperature below freezing. In implementations, the test sample is in a liquid state prior to the chilling and is in one of a frozen state, slurry state, or vitrified state subsequent to the chilling. In embodiments, the test sample is thawed from the frozen state, slurry state, or vitrified state to a thawed liquid state prior to detecting the color of the test sample.

In implementations, the determination of the anti-icing effects of the testing material is performed qualitatively based on a visual inspection of the color of the test sample. In some embodiments, the method includes detecting an initial color of the test sample prior to the chilling, wherein the determination of the anti-icing effects of the testing material is based on a comparison of: the initial color of the test sample prior to the chilling, and the color of the test sample subsequent to the chilling.

In some implementations, the detection of the color of the test sample is performed using spectroscopy or digital images, and the determination of the anti-icing effects of the testing material is performed quantitatively. In embodiments, the method includes the performance of a titration analysis including preparing serial dilutions of the test sample in a fixed ratio, with the ligand-grafted AuNP probe, thereby generating a plurality of different test samples. In such embodiments, the detection of the initial color of the test sample comprises detecting the initial color of each of the plurality of different test samples, and the detection of the color of the test sample subsequent to chilling comprising detecting the color of each of the plurality of different test samples subsequent to chilling. Next, a sigmoidal dose response curve is plotted based on differences between the initial color of each of the plurality of different test samples and the color of each of the plurality of different test samples subsequent to chilling, and a concentration of the testing material giving 50% of maximum efficacy (IC50) is determined based on an analysis of the sigmoidal dose-response curve, wherein the IC50 indicates a degree of the anti-icing effects of the testing material. In embodiments, the sigmoidal dose response curve is plotted based on one of: a change in surface plasmon resonance (SPR) at a select wavelength over a change in concentration of the test sample, and a change in aggregation factor (AF) over a change in concentration of the test sample for a select pair of wavelengths.

In some implementations, the dithiolate-based ligand has a bidentate binding group. In embodiments, the dithiolate-based ligand is modified with a terminal function group selected from the following: a carboxylic acid (COOH) group, a hydroxyl (OH) group, a methoxy (OCH₃) group, a sulfonate (SO₃⁻) group, an amine (NH₂) group, an azide (N₃) group, and a nitrilotriacetic acid (NTA) group. The dithiolate-based ligand may be modified with at least one terminal function group and an ethylene glycol moiety between a binding group and the at least one terminal functional group. In implementations, the dithiolate-based ligand is selected from: thioctic acid (TA), nitrilotriacetic acid modified thioctic acid (TA-NTA), oligo (ethylene glycol) modified thioctic acid (TA-OEG2), sulfonate modified thioctic acid (TA-SO₃⁻), and combinations thereof. In embodiments, the testing material comprises organic chemicals, inorganic chemicals or biomolecules. The method may further include the step of the dithiolate-based ligand-grafted gold nanoparticle (AuNP) probe in solution.

In another aspect of the invention, there is a colorimetric assay kit for determining anti-icing effects of a testing material comprising: a dithiolate-based ligand-grafted gold nanoparticle (AuNP) probe for mixing with test samples of a testing material in solution, wherein changes of color of the test samples after chilling indicate anti-icing effects of the testing material. In implementations, the colorimetric assay kit also includes a plurality of sample wells configured to house the respective test samples; and, optionally, a solution for diluting the testing material in the test samples. The dithiolate-based ligand may have a bidentate binding group. In embodiments, the dithiolate-based ligand is modified with a terminal function group selected from: a carboxylic acid (COOH) group, a hydroxyl (OH) group, a methoxy (OCH₃) group, a sulfonate (SO₃⁻) group, an amine (NH₂) group, an azide (N₃) group, and a nitrilotriacetic acid (NTA) group. In aspects of the invention, the dithiolate-based ligand may be modified with at least one terminal function group and an ethylene glycol moiety between a binding group and the at least one terminal functional groups. In some embodiments, the kit also includes instructions for determining anti-icing effects of the testing material using the colorimetric assay kit and a color chart comprising test sample colors with associated anti-icing indicia (e.g., IC50 indicia).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Aspects of the present invention are described in the detailed description which follows, in reference to the noted plurality of drawings by way of non-limiting examples of exemplary embodiments of the present invention.

FIG. 1A is an image comparing a solution containing AuNPs with antifreeze proteins (AFPs), and a solution containing AuNPs with an attached citrate ligand (Au-Cit), before and after freezing.

FIG. 1B is an image comparing three different control solutions after two hours at −20° C.

FIG. 2A is an illustrative representation of the theoretical Au-Cit clustering mechanism during freezing, wherein Au-Cit clusters are shown in a super-cooled region between ice crystals prior to thawing.

FIG. 2B is an illustrative representation of a clustering mechanism for Au-AFP during freezing, wherein Au-AFPs are shown dispersed throughout an Au-AFP solution between dispersed ice crystals, prior to thawing.

FIGS. 2C and 2D depict transmission electron microscope (TEM) images of the sample solutions of FIG. 1A after thawing.

FIG. 3A is a graph of absorbance versus wavelength for three AuNP solutions (Au-Cit, Au-MSA and Au-TA/NTA) in water before freezing.

FIG. 3B is a graph of absorbance versus wavelength for Au-Cit, Au-MSA and Au-TA/NTA solutions in a buffer before freezing.

FIG. 3C is a graph of absorbance versus wavelength for the Au-Cit, Au-MSA and Au-TA/NTA solutions of FIG. 3B, after freezing and thawing.

FIG. 3D shows the color of Au-Cit, Au-MSA and Au-TA/NTA test solutions, in both PBS and water, before freezing, while frozen, and after thawing.

FIG. 4A depicts the chemical structure of a first exemplary dithiolate ligand, thioctic acid (TA), for use in accordance with embodiments of the invention.

FIG. 4B depicts the chemical structure of a second exemplary dithiolate ligand, nitrilotriacetic acid modified thioctic acid (TA-NTA), for use in accordance with embodiments of the invention.

FIG. 4C depicts the chemical structure of a third exemplary dithiolate ligand, oligo (ethylene glycol) modified thioctic acid (TA-OEG₂), for use in accordance with embodiments of the invention.

FIG. 4D depicts the chemical structure of a fourth exemplary dithiolate ligand, sulfonate modified thioctic acid (TA-SO₃), for use in accordance with embodiments of the invention.

FIG. 5A is an image of a 96 well plate series filled with AuNP solutions with different types of target ligands mixtures and a control mixture, serially diluted (before freezing).

FIG. 5B is an image of the 96 well plate series of FIG. 5A after freezing.

FIG. 5C is an image of the 96 well plate series of FIG. 5B after thawing.

FIG. 6A is an image of a 96 well plate series titration test, utilizing an AuNP-TA/NTA probe in different test solutions at room temperature.

FIG. 6B is an image of the 96 well plate series titration test of FIG. 6A, after freezing for different freezing times (2 hours, 24 hours and 48 hours).

FIG. 6C is an image of the 96 well plate series of FIG. 6B, after thawing.

FIG. 7A is a graph showing UV-vis spectroscopy results (using a plate reader) of a solution containing an AuNP-probe and PVA (as a test material) before freezing (i.e., in an initial liquid state).

FIG. 7B is a graph showing UV-vis spectroscopy results (using a plate reader) of the solution of FIG. 7A after being frozen and then thawed (i.e., in a thawed state).

FIG. 7C is a graph showing a sigmoidal dose response curve depicting a change in SPR (ΔA₅₂₀) over a change in reagent concentration (Log[A] (mg/ml)) between the initial liquid state (FIG. 7A) and the thawed state (FIG. 7B).

FIG. 7D is a graph showing a sigmoidal dose response curve depicting a change in aggregation factor (ΔAF) over a change in reagent concentration (Log[A] (mg/ml)) between the initial liquid state (FIG. 7A) and the thawed state (FIG. 7B).

FIG. 7E is a graph showing UV-vis spectroscopy results (using a plate reader) of a solution containing an AuNP-probe and PVA (as a test material) before freezing (i.e., in an initial liquid state).

FIG. 7F is a graph showing UV-vis spectroscopy results (using a plate reader) of the solution of FIG. 7E after being frozen (i.e., in a frozen state).

FIG. 7G is a graph showing a sigmoidal dose response curve depicting a change in SPR (ΔA₅₂₀) over a change in reagent concentration (Log[A] (mg/ml)) between the initial liquid state (FIG. 7E) and the frozen state (FIG. 7F).

FIG. 7H is a graph showing a sigmoidal dose response curve depicting a change in aggregation factor (ΔAF) over a change in reagent concentration (Log[A] (mg/ml)) between the initial liquid state (FIG. 7E) and the frozen state (FIG. 7F).

FIG. 8A is an image of a 96 well plate series filled with AuNP-TA/NTA probe solutions with different regents, serially diluted (before freezing).

FIG. 8B is an image of the 96 well plate series of FIG. 8A after freezing (i.e., in a frozen state).

FIG. 8C is an image of the 96 well plate series of FIG. 8B after thawing (i.e., in a thawed state).

FIG. 9A is a graph showing a change in SPR (ΔA₅₂₀) over a change in reagent concentration (Log[A] (mg/ml)) for AuNP-TA/NTA probe solutions between an initial liquid phase and after being thawed from a frozen state (i.e., in a thawed state).

FIG. 9B is a graph showing a change in SPR (ΔA₅₂₀) over a change in reagent concentration (Log[A] (mg/ml)) for the AuNP-TA/NTA probe solutions between an initial liquid phase and a frozen state.

FIG. 9C is a graph showing a change in aggregation factor (ΔAF) over a change in reagent concentrations (Log[A] (mg/ml)) for the for the AuNP-TA/NTA probe solutions in an initial liquid phase and after being thawed from a frozen state (i.e., in a thawed state).

FIG. 9D is a graph showing a change in aggregation factor (ΔAF) over a change in reagent concentrations (Log[A] (mg/ml)) for the AuNP-TA/NTA probe solutions between an initial liquid phase and a frozen state.

FIGS. 10A-10F show images of IRI assay results for a plurality of test materials in buffers, for the purpose of comparison.

FIGS. 11A-11E show images of DIS assay results for a plurality of test materials in buffers, for the purpose of comparison.

FIG. 12 shows a flowchart of an exemplary AuNP-based HTS assay method in accordance with aspects of the present invention.

### DETAILED DESCRIPTION

Aspects of the present invention relate generally to colorimetric assays and, more particularly, to colorimetric assays using dithiolate-grafted nanoparticles (NPs) for high-throughput screening (HTS) of cryoprotectants or anti-icing materials.

In embodiments, a system and method are provided for high-throughput screening (HTS) of testing materials for anti-icing activities, across different molecular phases, using a stable dithiolate-grafted gold nanoparticle (AuNP) probe. Color changes of the dithiolate-grafted AuNP probe differ depending on the anti-icing efficacy or ice binding strength of the material being tested. In implementations, the color of a test sample, including a material to be tested and the dithiolate-grafted AuNP probe, changes as the temperature of the test sample changes over time, and indicates the anti-icing efficacy or ice binding strength of the test material.

A dithiolate-grafted AuNP probe according to embodiments of the invention has a high colloidal stability in solution with reversible aggregation of the AuNP probe after freezing and thawing, thus eliminating the need to adjust for a change in spectrum and color caused by post-thaw irreversibly aggregated AuNPs during a colorimetric assay. Embodiments of the invention use AuNPs of the stable dithiolate-grafted AuNP probes as a “molecular ruler” to measure distance between AuNP particles as a consequence of ice crystallization and ice recrystallization activity of co-solutes. In implementations, color changes are indicative of the distance between AuNPs, and are utilized to monitor the size of ice crystals formed over time. Additionally, aspects of the invention provide for a standardized test utilizing the AuNP probe, which can be replicated and utilized by different users to quantify and compare anti-icing test results.

Advantageously, a dithiolate-grafted AuNP probe according to embodiments of the invention demonstrates a much improved colloidal stability over AuNP alone or AuNP-MSA, and can be utilized with various types of biochemical buffers and target materials without pre-aggregation of the AuNP. This is a significant improvement over previous methods that are limited to certain buffers, concentrations of ions, or pH, necessary for various types of testing reagents. In implementations, the colorimetric assays of the present disclosure enable the monitoring of anti-icing activity in different phases or states (e.g., liquid, slush, vitrified, or solid) at different temperatures, and/or in different environments, by (1) qualitatively observing color changes, (2) utilizing a measurement of spectroscopy, or (3) using a direct color image analysis, each of which may be quickly performed. Further, a colorimetric assay in accordance with this disclosure can be utilized to determine anti-icing effects utilizing a serial dilution-based titration method and half-maximal inhibitory concentration (IC50) calculation based on a dose-response curve fitting (e.g., using the Hill equation). This enables a standardized quantified efficacy measure of anti-icing activity that may be easily compared to results from different experimental conditions or batches.

### 1.0 Design of AuNP Probe for HTS Assay Development

### 1.1 AuNP for Detecting Anti-Icing Molecules as a Molecular Ruler

In general, colorimetric assays use a probe configured to bind a target material or molecule, wherein binding of the target material by the probe results in a color change. AuNP has a strong surface plasmon resonance (SPR) property that is sensitive not only to chemical binding at the surface of the AuNP, but also to nanoparticle-to-nanoparticle interactions (via plasmon coupling), resulting in a SPR peak shift or colloidal color change depending on the strength (or distance) of the interaction. Thus, AuNP is a common probe for NP-based colorimetric assays to sense many types of target materials including simple ions, as well as specific interactions between biomolecules such as DNA-DNA hybridization and antibody-antigen interaction.

In general, it is undesirable to freeze AuNP due to the resulting loss of intrinsic properties (such as color, emission, size and shape) because of the tendency of the nanoparticles to aggregate when they are frozen, and remain aggregated after thawing.

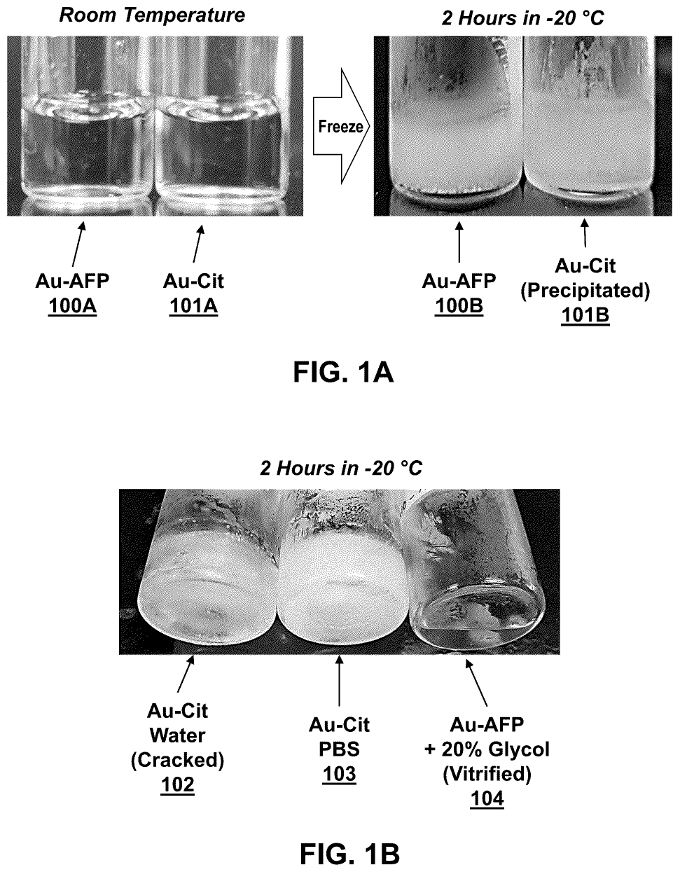

FIG. 1A is an image comparing a solution containing AuNPs with antifreeze proteins (AFPs), and a solution containing AuNPs with an attached citrate ligand (Au-Cit), before and after freezing. More specifically, a room-temperature (liquid state) sample solution 100A of AFPs and AuNPs (Au-AFP) is indicated at 100A, and a room-temperature (liquid state) sample solution of Au-Cit is indicated at 101A. In the example of FIG. 1, the AuNPs utilized were 10 nm in diameter. Both liquid solutions 100A and 101A demonstrated a light red colloidal color. After freezing the solutions 100A and 101A at −20° C. for 2 hours, the resulting sample solutions 100B and 101B are depicted in a frozen state. In the frozen state, the colloidal color of sample 101B was completely lost and became almost a translucent grey color. The mechanism that explains this loss in color, in theory, is that ice crystals push any impurity out of the pure ice crystal during the freezing of the solution, which makes nanoparticles concentrate in the super-cooled region in-between ice crystals. Over time in freezing temperatures, highly concentrated nanoparticles in a local area between ice crystals aggregate and lose their mobility with a decreasing diffusion constant. Contrarily, the colloidal color of sample 100B stayed red after freezing, indicating that Au-AFP prohibited the growth of large ice crystal formation and stayed dispersed (instead of aggregating together in the frozen state).

FIG. 1B is an image comparing three different control solutions after two hours at −20° C. More specifically, a first control solution 102 comprising Au-Cit in water is in a frozen state that includes visible cracks in the frozen solution. A second control solution 103 comprises Au-Cit in phosphate-buffered saline (PBS), and is in a frozen state without cracking but with loss of red color due to aggregation. A third control solution 104 comprises Au-AFP in 20% glycol, and is in a vitrified state (between a liquid state and a solid state) with a red color as a consequence of no ice formation and AuNP aggregation.

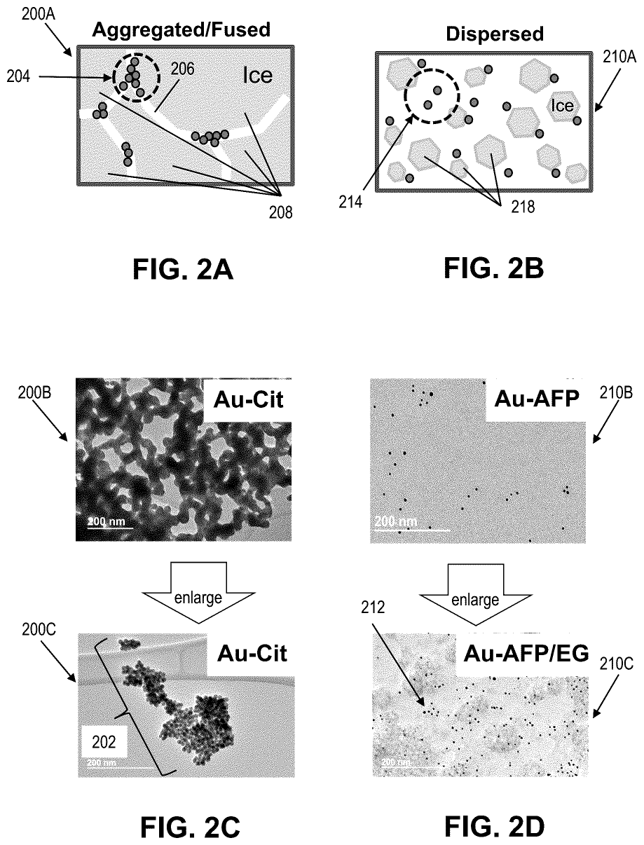

FIG. 2A is an illustrative representation of the theoretical Au-Cit clustering mechanism during freezing, as depicted at 200A, wherein Au-Cit clusters (e.g., 204) are shown in super-cooled region 206 between ice crystals 208 prior to thawing.

FIG. 2B is an illustrative representation of a clustering mechanism for Au-AFP during freezing, as depicted at 210A, wherein Au-AFPs (e.g., 214) are shown dispersed throughout an Au-AFP solution between dispersed ice crystals (e.g., 218), prior to thawing.

FIGS. 2C and 2D depict transmission electron microscope (TEM) images of the sample solutions of FIG. 1A after thawing. Often, the aggregation of AuNP during freezing is irreversible so that AuNP (e.g., citrate capped AuNP) does not re-dispersed after thawing. This aggregated state was confirmed by the images of FIG. 2C.

More specifically, FIG. 2C shows an image 200B of the sample 101B of FIG. 1 after thawing. An enlarged portion of the image 200B is shown at 200C, depicting clusters of Au-Cit at 202. If there is an anti-icing or ice-inhibiting reagent in the AuNP solution that has some level of binding or interaction to the ice, ice growth can be inhibited and become smaller to offer a much larger super-cooled region, as well as a much higher surface-to-volume ratio in ice (compared with those of large ice without an anti-icing reagent), which eventually helps nanoparticles disperse and be less aggregated. See the illustration of FIG. 2B, for example. Depending on the anti-icing efficacy or ice binding strength, the color change of AuNP solutions due to aggregation or plasmon coupling is different (see the Au-AFP in FIG. 1A), or is not changed at all, even after being frozen for the case of vitrification (see the Au-AFP+20% Glycol in FIG. 1B).

With reference to FIG. 2D, an image 210B is shown of the sample 100B of FIG. 1 after thawing. An enlarged portion of the image 210B is shown at 210C, depicting dispersed nanoparticles (e.g., at 212) in an Au-AFP solution with 20% ethylene glycol (EG).

Image 210C in FIG. 2D confirms that the Au-AFP solution 100A with 20% EG was well dispersed to keep large distances between AuNPs, indicating that a full recovery of the test sample after thawing, without any aggregation of the AuNPs, is well correlated with no color change during a frozen state, as depicted in FIG. 1A. Indeed, the aggregation of AuNPs caused by plasmon coupling between AuNPs, due to changes in distance between the AuNPs, can be used for detecting an anti-icing effect of a reagent. This “molecular ruler” activity may be an important factor in estimating the apparent (or averaged) distance between AuNPs to measure the efficacy of target reagents in different phases (e.g., liquid, frozen, or mixed).

### 1.2 Stability Test of Thioctic Acid-Based AuNPs

Colloidal AuNP easily aggregates with small changes of pH, ionic concentration or other chemical binding, as well as temperature, which may result in false signals (without the addition of target molecules), and is therefore not desirable for any type of colorimetric assay. To improve intrinsic colloidal stability for a robust AuNP probe, the surface of the AuNP probe can be modified using different types of chemicals, referred to as a “ligand” herein. One of the most common ligands for use with AuNPs is citrate, but it does not stabilize AuNPs in low pH or high ionic conditions due to the weak interaction between a carboxyl group (—COOH) of citrate and an Au atom on the surface of the AuNPs, and the small physical size of citrate. Based on the above, a ligand of choice for higher stability is a thiolate-based ligand due to a strong interaction between sulfur(S) in a thiol group (—SH) and Au. If the ligand has a stronger binding group (e.g., thiol) or multivalent binding groups (e.g., dithiol or disulfide), the colloidal stability and solubility of nanoparticles in a buffer can be improved dramatically.

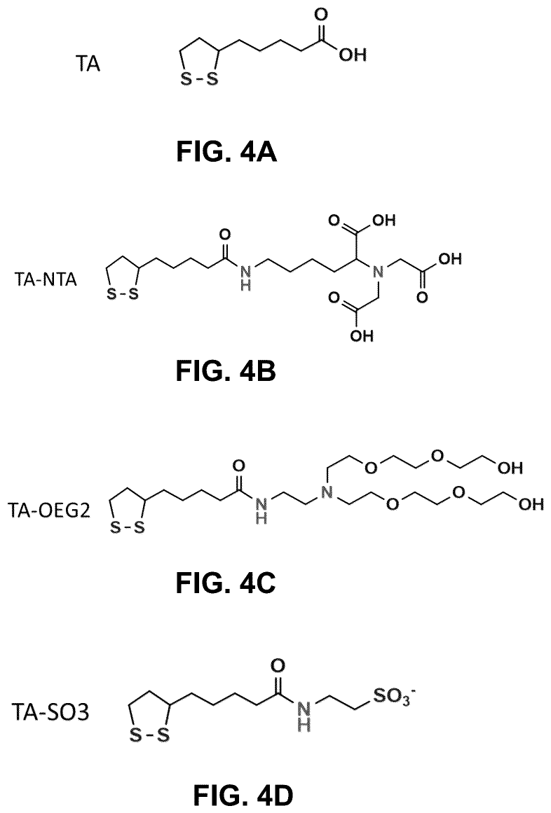

Testing discussed herein utilized the simplest structure of dithiolate ligands, thioctic acid (TA) and thioctic acid with nitrilotriacetate (TA-NTA), to determine their response to the frozen state in terms of AuNP's aggregation at a post-thaw state (after a frozen state). Both TA and TA-NTA showed great color changes before and after freezing, but TA was more sensitive to temperature changes. Since TA-NTA has three COOH groups with a longer size, it was theorized that the colloidal stability of TA-NTA would be improved over the colloidal stability of TA itself. See FIG. 4B, depicting the structure of TA-NTA. To optimize a response and stability, these two ligands were mixed to develop a TA-NTA+AuNP (Au-TA/NTA) probe for colorimetric assay.

First, the stability of the Au-TA/NTA probe was tested and compared to AuNP modified with MSA (Au-MSA) and Au-Cit in ionic buffer conditions using PBS. PBS is a buffer solution with pH˜7.4, and 1×PBS has a final concentration of 157 millimols (mM) Na⁺, 140 mM Cl⁻, 4.45 mM K⁺, 10.1 mM HPO₄²⁻, 1.76 mM H₂PO₄⁻ and a pH of 7.96. Hydrochloric acid (HCl) was added at 2.84 mM to shift the buffer to 7.3 mM HPO₄²⁻ and 4.6 mM H₂PO₄⁻ for a final pH of 7.4 and a Cl⁻ concentration of 142 mM (commonly used in biological research). AuNPs at 10 nm were synthesized in a first batch with citrate, a second batch was surface modified with MSA, and a third batch was surface modified with TA-based ligands (e.g., TA/NTA=mixed with 99% of TA and 1% of TA-NTA). To complete the modification reaction, an excessive amount of ligand (e.g., 2500 times higher amount of ligand than Au molecules) was used, keeping the reaction going overnight (e.g., at least 8 hours) with vigorous stirring, and the modified AuNP samples were purified using a centrifuge membrane filter (e.g., 10K Molecular weight cut-off) to remove unbound ligands as well as original reagents to be used for synthesis of AuNP. The colloidal stability of the resulting Au-Cit, Au-MSA and Au-TA/NTA were then tested in high salt biological buffer conditions (1×PBS as a final concentration after mixing with 10 nM of AuNP) before and after freezing.

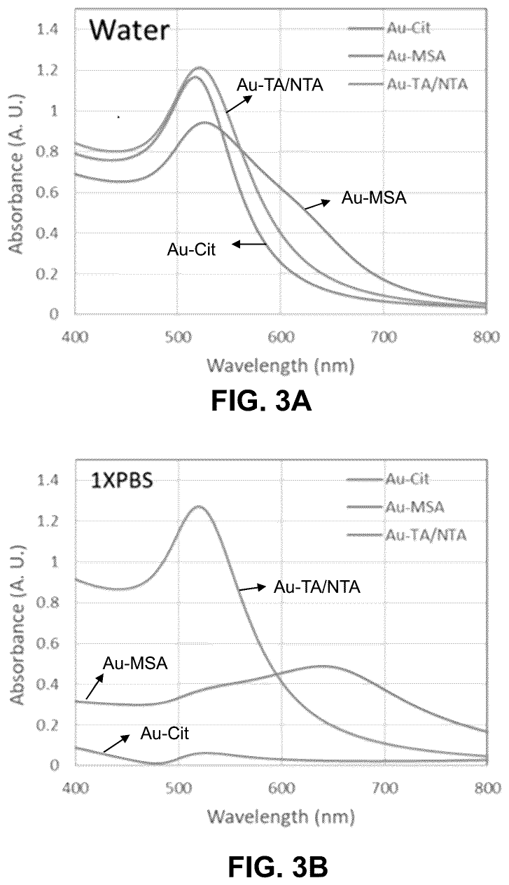

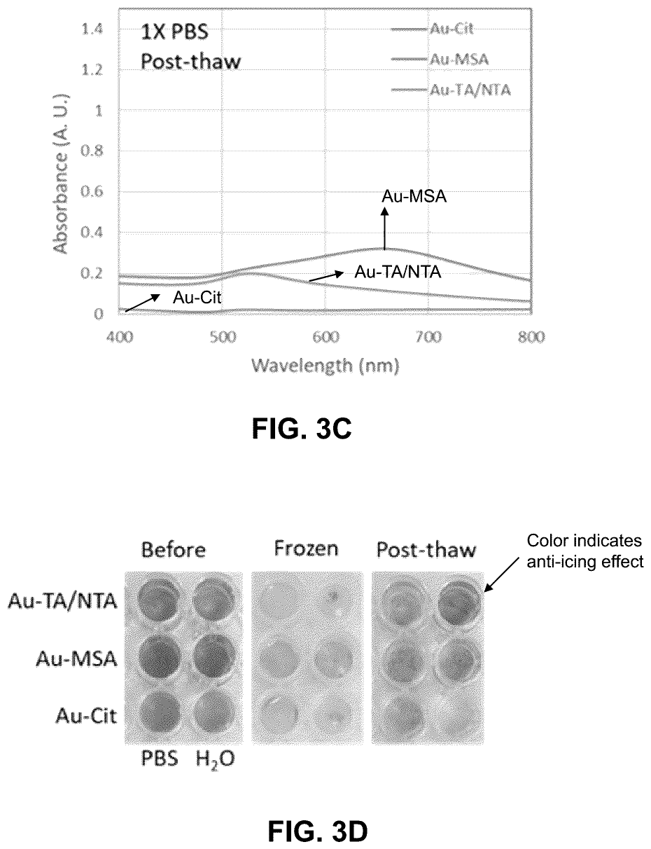

FIG. 3A is a graph of absorbance versus wavelength for three AuNP solutions (Au-Cit, Au-MSA and Au-TA/NTA) in water before freezing. More specifically, FIG. 3A compares results of an ultraviolet-visible (UV-Vis) spectroscopy analysis of Au-Cit, Au-MSA and Au-TA/NTA in water (H₂O). The results illustrate that the UV-vis absorption spectrum of Au-TA/NTA in water and Au-MSA showed small changes in spectrum with respect to Au-Cit, indicating that there is a small change in SPR.

FIG. 3B is a graph of absorbance versus wavelength for Au-Cit, Au-MSA and Au-TA/NTA solutions in a buffer before freezing. Specifically, FIG. 3B illustrates the spectrum of each AuNP with different ligands in a 1×PBS buffer after freezing at −20° C. for 2 hours. Au-Cit and Au-MSA showed SPR shifts from an original peak (˜520 nm) to a longer wavelength (˜650 nm), or loss of absorption in SPR at 520 nm (e.g., flat spectrum) due to aggregation, while Au-TA/NTA showed little or no changes. This result demonstrated that citrate and MSA are not as stable as the TA-based ligand, and can generate false signals when used to test biomolecules or cell extractions in a PBS buffer.

Next, freezing conditions of Au-Cit, Au-MSA, and Au-TA/NTA solutions were tested to confirm the use of AuNPs for colorimetric assays. The Au-Cit, Au-MSA, and Au-TA/NTA solutions were frozen at −20° C. for 2 hours and thawed at room temperature for 30 minutes.

FIG. 3C is a graph of absorbance versus wavelength for the Au-Cit, Au-MSA and Au-TA/NTA solutions of FIG. 3B, after freezing and thawing. As illustrated in FIG. 3C, Au-Cit did not show much of a change between molecular phases, since it already lost SPR before freezing. Au-MSA and Au-TA/NTA both demonstrated irreversible aggregation and lost their colloidal dispersity permanently, resulting in significant changes of the spectrum and color at the moment of post-thaw. Interestingly, Au-TA/NTA has the most change in color, up to 85% of original absorbance at 520 nm (A₅₂₀) during the frozen state, while Au-MSA has a change of up to 40% of the original absorbance at 520 nm, implying that Au-TA/NTA is the most beneficial to develop sensing systems with high dynamic ranges. Thus, Au-TA/NTA demonstrated superior colloidal stability when compared to Au-MSA or Au-Cit, especially in general biological buffer conditions.

FIG. 3D shows the color of Au-Cit, Au-MSA and Au-TA/NTA test solutions, in both PBS and water, before freezing, while frozen, and after thawing. More specifically, FIG. 3D illustrates the colloidal color changes that are consistent with the detected spectrum change of Au-Cit and Au-MSA in different buffers (i.e., PBS and water). The red color in the thawed Au-TA/NTA sample indicates that TA/NTA has an anti-icing effect.

### 1.3 Ligand Effect on the Freezing of AuNP

Next, the role of ligands in a colorimetric assay for a frozen sample test was investigated, and appropriate concentrations were found to optimize an AuNP probe to test anti-icing activity. An AuNP probe (using the same method as the previous stability testing section) was used to test how four different types of ligands (TA, TA-NTA, TA-OEG₂ and TA-SO₃⁻) affect AuNP during freezing.

FIG. 4A depicts the chemical structure of a first exemplary dithiolate ligand, thioctic acid (TA), for use in accordance with embodiments of the invention. TA is commercially available, such that the synthesis of TA will not be discussed in detail herein.

FIG. 4B depicts the chemical structure of a second exemplary dithiolate ligand, nitrilotriacetic acid modified thioctic acid (TA-NTA), for use in accordance with embodiments of the invention. TA-NTA synthesis has been discussed in the literature, and will not be discussed in detail herein.

FIG. 4C depicts the chemical structure of a third exemplary dithiolate ligand, oligo (ethylene glycol) modified thioctic acid (TA-OEG₂), for use in accordance with embodiments of the invention. A synthesis method is discussed in detail below.

FIG. 4D depicts the chemical structure of a fourth exemplary dithiolate ligand, sulfonate modified thioctic acid (TA-SO₃⁻), for use in accordance with embodiments of the invention. TA-SO₃⁻ synthesis has been discussed in the literature, and will not be discussed in detail herein.

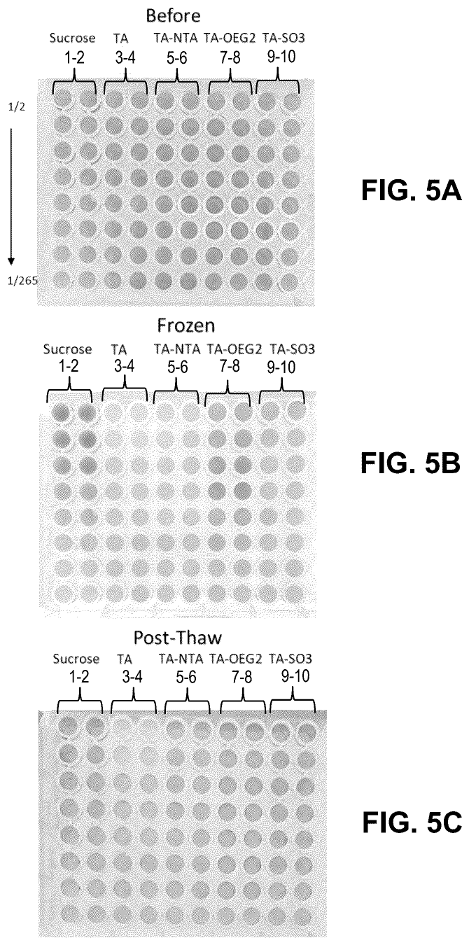

In a sample well plate series (96 wells), a 2 millimolar (mM) stock solution of the test ligands was mixed with AuNP, resulting in a 1 mM AuNP-probe solution which was deposited as a highest concentration in respective top wells (using two columns or lanes of wells for each ligand), and was serially diluted by half using 2×PBS buffer for every following well. More specifically, the stock ligand concentration, 1 mM, was diluted by ½, ¼, ⅛, 1/16, 1/32, 1/64, 1/128, and 1/256 times (from top to bottom) by using 2×PBS. After finishing serial dilution of the target materials (four ligands, here), 50 microliters (μL) of 20 nM AuNP were added to each well of the well plate series to make a 10 nM AuNP-probe in 1×PBS in each well as a final concentration. Sucrose 40% was also used as a control target material to confirm that the AuNP-based colorimetric assay was responding to the serial dilution of sucrose by using the same titration method. See the resulting sample well plate series in FIG. 5A.

FIG. 5A is an image of a 96 well plate series filled with AuNP solutions with different types of target ligands mixtures and a control mixture, serially diluted (before freezing). FIG. 5A shows that all AuNPs samples look well-dispersed without any color changes in the presence of the test ligands TA, TA-NTA, TA-OEG₂ and TA-SO₃⁻, as well as sucrose as a control. The solutions in the well plate series were then frozen at −20° C. for a desired time. See FIG. 5B.

FIG. 5B is an image of the 96 well plate series of FIG. 5A after freezing. Specifically, the 96 well plate series was frozen at −20° C. for 2 hours, resulting in the frozen state of the samples depicted in FIG. 5B. The frozen samples showed dynamic changes of colloidal color depending on ligand type and concentration. In the presence of sucrose (lanes 1-2), the colloidal color of AuNP changed from the original red (top well, at the highest concentration) to purple, and finally to pale purple with the decrease in sucrose concentration, which confirmed that a colorimetric assay using the AuNP probe was very responsive to the concentration of a target material for anti-icing activity. The colloidal color of AuNP with TA (lanes 3-4) or TA-NTA (lanes 5-6) turned translucent at the highest concentrations, indicating AuNP aggregation, while AuNP with TA-OEG₂ (lanes 7-8) TA-SO₃⁻ (lanes 9-10) was red at the highest concentration without any aggregation even during the frozen state.

FIG. 5C is an image of the 96 well plate series of FIG. 5B after thawing. More specifically, the 96 well plate series of FIG. 5B was thawed at room temperature for 30 minutes, resulting the post-thaw state of FIG. 5C. After thawing, the colloidal color of AuNP with only TA remained pale at the highest concentration. See lanes 3-4 of FIG. 5C. AuNP with TA-NTA and TA-SO₃⁻ showed SPR changes only during the frozen state (see lanes 5-6 and 9-10 of FIG. 5B), and almost fully recovered their intrinsic optical properties at the post-thaw state (see lanes 5-6 and 9-10 in FIG. 5C), indicating that they have high colloidal stability with reversible aggregation only for the frozen state. AuNP with TA-OEG₂ turned from red to pale red with decreases in ligand concentration during the frozen state (see lanes 7-8 in FIG. 5B) and recovered to almost the original red color at the post-thaw state (see lanes 7-8 in FIG. 5C).

In conclusion, TA is a very useful ligand for monitoring anti-icing activity for both frozen and post-thaw states of samples due to irreversible aggregation, and TA-NTA and TA-SO₃⁻ are useful to monitor samples during a frozen state due to reversible aggregation. Interestingly, TA-OEG₂ was found to act almost like a protection ligand for AuNP during a frozen state.

### 1.4 Different Phase, Temperature and Time-Resolved HTS Test

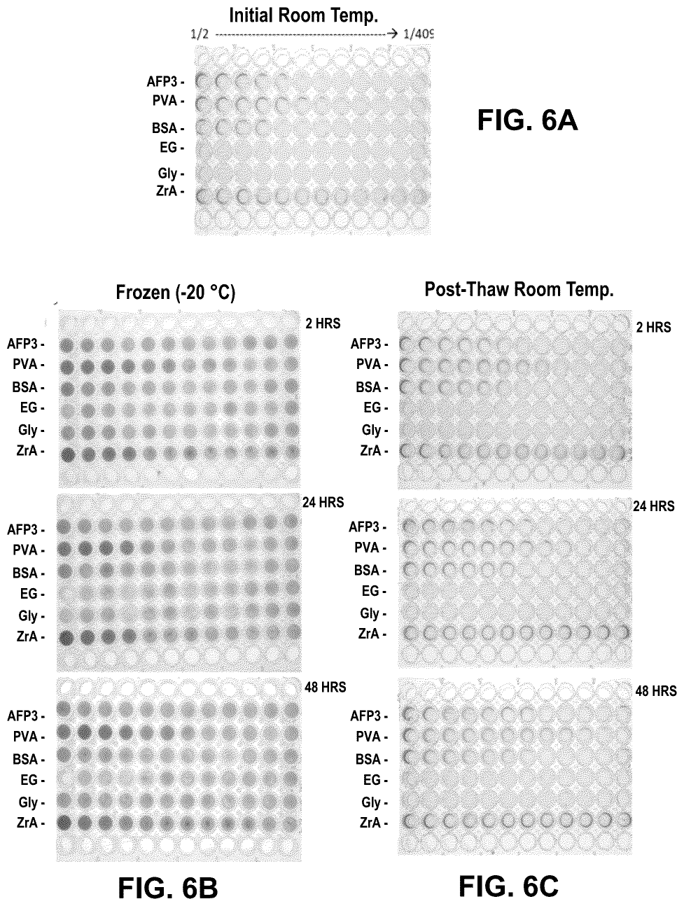

Implementations of the invention utilize AuNP for monitoring different physical phases (liquid and solid) of an analyte solution to test for anti-icing effects. It was determined during the freezing test discussed in the previous section that the frozen state of the sample can be useful. To date, it does not appear that this insight has been addressed or utilized, potentially due to the lack of understanding and a lack of an analysis tool for this frozen state of AuNPs. For the purpose of anti-icing effect testing, it is reasonable to compare the “frozen state” of targets. To confirm this hypothesis, six different known anti-icing materials were tested using an AuNP-TA/NTA probe, as illustrated in FIGS. 6A-6C.

FIG. 6A is an image of a 96 well plate series titration test, utilizing an AuNP-TA/NTA probe in different test solutions at room temperature. From top to bottom there are six different testing targets: (1) AFP 0.3 mg/ml, (2) polyvinyl alcohol (PVA) 10K 0.3%, (3) bovine serum albumin (BSA) 0.3 mg/ml, (4) ethylene glycol (EG) 50%, (5) glycerol (Gly) 50%, and (6) 80 mg/ml zirconium acetate (ZrA). The buffer was 1×PBS except for the last sample ZrA, which was in 40 mM HCl (because ZrA is only active in acidic conditions). The target samples were diluted in rows by ½, ¼, ⅛, 1/16, 1/32, 1/64, 1/128, 1/256, 1/512, 1/1024, 1/2048 and 1/4096 times (from left to right) using 2×PBS or 80 mM HCl (for ZrA). Next, 50 μL of target materials were mixed with 50 μL of 20 nM AuNP-probe to make a final 1×PBS, or 40 mM HCl for the last sample, and 10 nM of AuNP probe in each well.

FIG. 6B is an image of the 96 well plate series titration test of FIG. 6A, after freezing. More specifically, the 96 well plate series of FIG. 6A was frozen for 2 hours at −20° C., for 24 hours at −20° C., and for 48 hours at −20° C.

FIG. 6C is an image of the 96 well plate series of FIG. 6B, after thawing. Initially, it was confirmed that AuNP-TA/NTA probes of the invention discussed herein can be used for different phases of anti-icing measurements. The frozen state, or solid phase, of AuNP-TA/NTA demonstrated a color change depending on the concentration of target materials (see FIG. 6B), as well as a liquid state at post thaw (see FIG. 6C).

Second, it was found that the frozen state showed consistently repeated results after longer freezing times as well as repeated freezing-thaw cycles (see FIG. 6B), indicating that this frozen state was actually reflecting the intrinsic properties of each sample. More importantly, since color of an AuNP colloid was also affected by the plasmon coupling between the nearest AuNP, color changes can be an indication of a distance between AuNP as a collective result. If it is assumed that the distance between AuNPs can be used to monitor the size of ice crystals, based on the premise that ice crystals push away impurities including AuNPs and make the concentration of AuNPs higher in-between ice crystals (thereby pushing the AuNP closer together), we can use AuNPs as a “molecular ruler” to measure distance between AuNPs as a consequence of ice crystallization and ice recrystallization activity of co-solutes.

In implementations, the portion of AuNP-clusters can be extrapolated from the total spectrum using theoretical Mie theory, and the apparent mean distance between AuNPs can be calculated. Based on Mie theory, clustering of AuNP will show different profiles of absorption spectrum, and the expected spectrum can be theoretically simulated from each type of clustered AuNPs (e.g. single AuNP, 2 clusters of AuNPs and 20 clusters of AuNPs separately). As an example, when using the total spectrum of AuNP-probe freezing test in the presence of PVA 1.5 mg/ml to extract the portion of AuNPs aggregations, the cluster portion was 67% of single AuNP+28.4% of 2 clusters+5% of 20 clusters in total volume of 100 μL of 10 nanomoles (nM) of 10 nm AuNP-probe and the apparent mean distance was approximately 289 nm.

### 1.5 Quantification of AuNP-Based HTS Assay

Implementations of the invention may utilize different color detection methods, both qualitative and quantitative. In general, a qualitative color analysis may be performed by a user simply observing the color of a test sample after freezing (e.g., in a frozen state) or in a thawed state. For example, a test sample that is red in color indicates the presence of an anti-icing material.

Quantitative color detection methods may utilize spectroscopy or digital image analysis to determine anti-icing activities of testing materials. In one example, digital color images of samples may be taken, and a color image analysis may be performed by a computer processor(s) to produce a quantifiable color result. For example, the color of a negative control sample may be compared to a color of a test sample using digital image colorimetry (DIC). Various digital-image analysis methods may be utilized in accordance with embodiments of the invention, and the invention is not intended to be limited to only the examples described herein.

In the case of spectroscopy, wavelength data may be used in accordance with implementations of the invention to generate an IC50 number that represents a testing material's anti-icing effect. In embodiments, tracking of changes in surface plasmon resonance (SPR) and/or the aggregation factor (AF) over molecular phases have been used for identifying IC50 numbers of testing materials. In one example, the ratio of absorbance of a test sample at 520 nm to the absorbance of the test sample at 650 nm is represented as “A520/A650” (referred to herein as the aggregation factor), and may be used to compare the amount of light absorbed by a sample at these two specific wavelengths to analyze the aggregation state of nanoparticles.

SPR represents the size effect of a single AuNP as well as interactions between other molecules (co-solutes) and the AuNP. If there is aggregation, (1) the SPR peak shifts to a longer wavelength, (2) absorbance is lost at the original SPR position, and (3) absorbance at longer wavelengths (e.g., at 650 nm or beyond) increases. Thus, the AF can indicate the effect of aggregation and reflect the shape change of spectrum induced by aggregation. These are common analysis methods for colorimetric assays. However, some reagents induce AuNP aggregates in a buffer, resulting in SPR and aggregation factor changes compared to the intrinsic AuNP itself. In such situations, the SPR and aggregation factors are not useful or accurate indicators of anti-icing factors.

As a solution to the above-identified problem, the Applicant utilized changes in the SPR and the aggregation factor (AF) of a test solution, before and after freezing of the test solution, as an analysis factor to remove the effect of aggregation due to mixing AuNP with certain reagents before freezing.

The following first equation Eq (1) represents the AF, wherein A₅₂₀ is the absorbance of a test solution at 520 nm and A₆₅₀ is the absorbance of the test solution at 650 nm:

$$AF = \frac{{A}_{520}}{{A}_{650}} . \qquad Eq ( 1 )$$

Here, A₅₂₀ and A₆₅₀ indicate the absorbance of AuNP at 520 nm and 650 nm respectively, after baseline correction by subtracting the buffer spectrum and the absorbance at the lowest wavelength in the whole spectrum collected (e.g. A₁₁₀₀ for cuvette-based UV-vis spectroscopy).

The following is a second equation Eq (2) representing the change in AF (ΔAF), wherein AF (before) represents the aggregation factor before the test solution is frozen, and AF (after) represents the aggregation factor after the test solution has been frozen:

$$Δ AF = AF ( before ) - AF ( after ) . \qquad Eq ( 2 )$$

The following is a third equation Eq (3) representing the change in SPR (ΔA₅₂₀) at the wavelength of 520 nm before and after the freezing process, wherein A₅₂₀ (before) is the absorbance of AuNP at the wavelength of 520 nm before the test solution is frozen and A₅₂₀ (after) is the absorbance of AuNP at the wavelength of 520 nm after the test solution is frozen.

$$Δ {A}_{520} = {A}_{520} ( before ) - {A}_{520} ( after ) . \qquad Eq ( 3 )$$

Based on the above, the Applicant utilized the changes of the SPR (ΔA₅₂₀=A₅₂₀ (before)−A₅₂₀ (after)) and changes of the aggregation factor (ΔAF=AF (before)−AF (after)) before and after a freezing process as an analysis parameter, to remove the effect of aggregation happening by mixing a reagent with AuNP before freezing. This gap or difference is now solely reflecting “the loss of single AuNP” (SPR change, ΔA₅₂₀) or “the loss of monodispersity” (ΔAF) due to the freezing process.

Additionally, the Applicant suggests a more robust analysis method to quantify colorimetric results and compare the colorimetric results of different tests or different test batches. More specifically, the Applicant presents a novel titration method to detect anti-icing activity using AuNP-based colorimetric assay methods presented herein, instead of a single concentration-based comparison.

In general, titration is a common laboratory method for quantitative chemical analysis to determine the concentration of an identified analyte in terms of activity. In accordance with implementations of the invention, a novel titration method includes performing serial dilutions on a test sample in a fixed ratio (e.g., such as 1:1, 1:2, 1:4, 1:8, etc.) until the last dilution does not give a positive test for the presence of analytes. Often different materials require different dynamic ranges and sensitivities that can be confirmed during this method, and finally used for modification of AuNP for proper detection of target materials. Thus, it is necessary to obtain a quantified value or parameter in order to compare different test results in different conditions. By using a titration analysis, often we can see the sigmoidal dose-response curve as a result and can quantify the efficacy of anti-icing effects based on a colorimetric assay to compare each reagent or test sample for: (1) maximum activity, (2) IC50 (the concentration of analyte giving 50% of maximum efficacy), and (3) a slope of a reactivity curve in assay that shows the interaction between a target and reagent.

In embodiments, the following equation Eq (4) (the Hill equation), is used to analyze a dose-response curve for a reagent of interest, wherein E is the response of the reagent during assay, n is the slope of the plot (i.e., Hill coefficient), E_{Max} is the maximal response, IC50 is the reagent concentration that produces a 50% of maximal response, and [A] is the concentration of the reagent:

$$E = \frac{{E}_{Max}}{1 + {( \frac{{IC}_{50}}{[ A ]} )}^{n}} . \qquad Eq ( 4 )$$

The Hill equation is commonly used for protein-ligand interactions or dose-response curves in drug studies, but has not been utilized in conjunction with AuNP-based anti-icing tests. A slope, n, greater than one indicates positively cooperative binding between a target and a reagent (i.e., once one molecule is bound to the target, its affinity for other molecules increases), while a slope less than one indicates negatively cooperative binding (i.e., once one molecule is bound to the target, its affinity for other molecules decreases). Thus, the titration method discussed herein and Hill equation are applied to AuNP-based colorimetric assays in accordance with embodiments of the invention, and can be useful to analyze the results of the colorimetric assay and to apply Applicant's understanding to the interaction between anti-icing materials and ice.

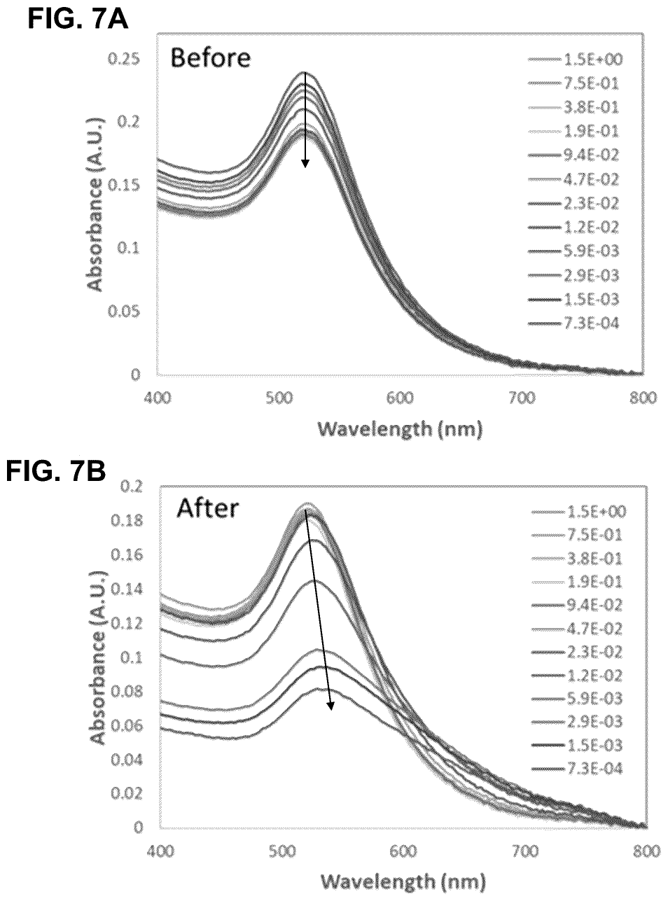

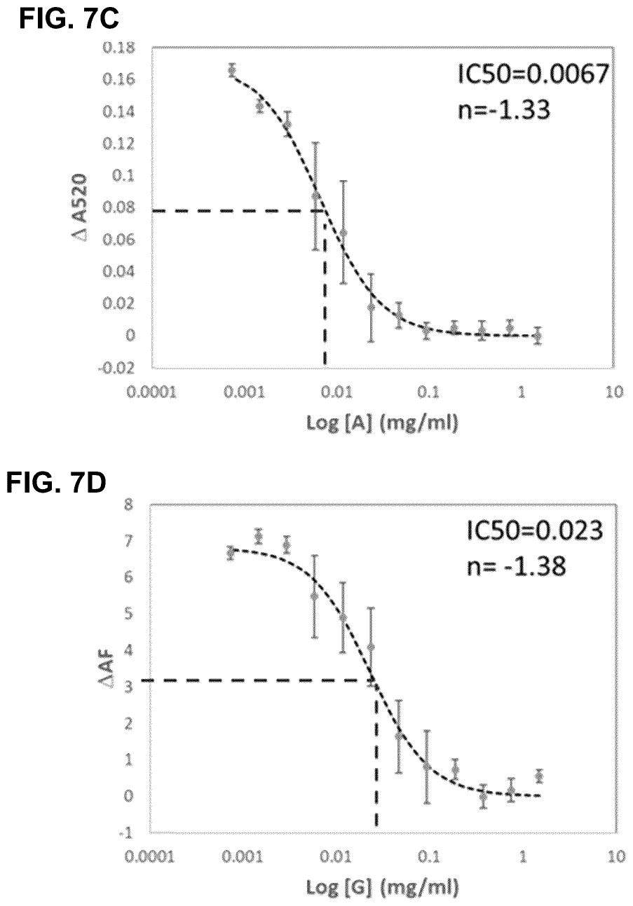

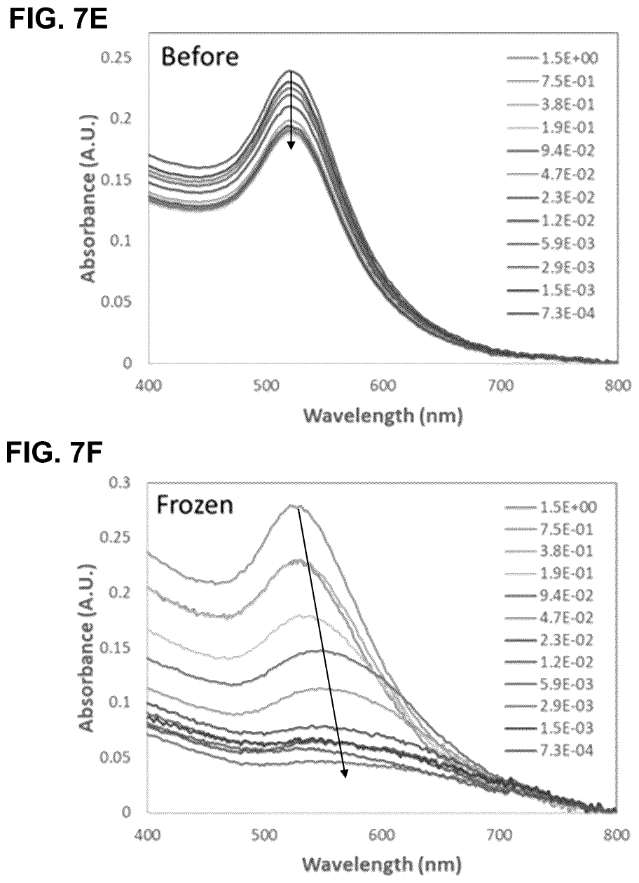

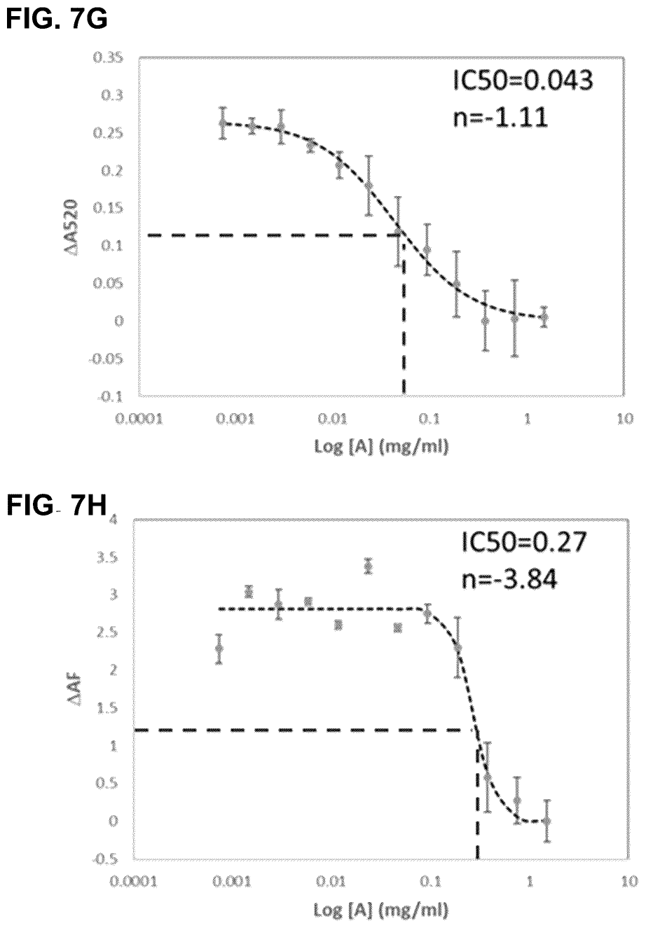

There are two types of data collection that may be utilized for the IC 50 analysis according to embodiments of the invention: (1) spectroscopic data collection and (2) corresponding color images. Exemplary spectroscopic data collection methods include collecting UV-vis spectroscopy data of a well plate series (e.g., a 96-well plate series) by cuvette-based spectroscopy (e.g., using cuvette's by Hamamatsu, Inc.), or by a plate reader (e.g., a Spark® Multimode Microplate Reader by Tecan Trading AG) to collect baseline corrected absorbance at various wavelengths (see, e.g., FIG. 7A). Exemplary color image collection methods include capturing a color camera image of a well plate series (e.g., 96 well plate series) at each moment or different phase of samples (i.e., before, frozen, and post-thaw). Examples of such colored images are shown in FIGS. 5A-5C. Advantageously, the use of such color image collection methods enables the quick and easy collection of data at any given moment, which is necessary for HTS tests. Additionally, the Applicant suggests that the quantified values from assay, “IC50” and “n”, derived from the Hill equation fitting of experimental results, are reflected in the efficacy of anti-icing materials and can be used as standardized values to compare to other experiments or other batches of the same experiment.

In implementations of the invention, for both the spectroscopic method and the color imaging method, the Hill equation (Eq (4)) is used to calculate the efficacy of anti-icing. As an example, this analysis method was utilized for the analysis of an AuNP-TA/NTA probe and PVA test solution, as set forth in FIGS. 7A-7H. More specifically, spectrum changes were collected for a before freezing state (liquid state), thawed state, and frozen state, and the results in terms of changes in absorbance and changes in surface plasmon resonance were plotted as sigmoidal dose-response curves.

FIG. 7A is a graph showing UV-vis spectroscopy results (using a plate reader) of a solution containing PVA (as a test material) before freezing (i.e., in an initial liquid state). More specifically, FIG. 7A shows a graph of absorbance (A.U.) versus wavelengths (nm) for a solution including AuNP-TA/NTA in PVA.

FIG. 7B is a graph showing UV-vis spectroscopy results (using a plate reader) of the solution of FIG. 7A after being frozen and then thawed (i.e., in a thawed state). It can be seen that the absorbance distribution of the solution containing PVA after thawing is greater than the absorbance distribution before freezing, as illustrated by the arrows in FIGS. 7A and 7B.

FIG. 7C is a graph showing a sigmoidal dose response curve depicting a change in SPR (ΔA₅₂₀) over a change in reagent concentration (Log[A] (mg/ml)) between the initial liquid state (FIG. 7A) and the thawed state (FIG. 7B). A resulting IC50 of 0.0067 and a slope n of −1.33 was then calculated.

FIG. 7D is a graph showing a sigmoidal dose response curve depicting a change in AF (ΔAF) over a change in reagent concentration (Log[A] (mg/ml)) between the initial liquid state (FIG. 7A) and the thawed state (FIG. 7B). A resulting IC50 of 0.023 and a slope n of −1.38 was then calculated.

FIG. 7E is a graph showing UV-vis spectroscopy results (using a plate reader) of a solution containing PVA (as a test material) before freezing (i.e., in an initial liquid state). More specifically, FIG. 7E shows a graph of absorbance (A.U.) versus wavelengths (nm) for a solution including AuNP-TA/NTA in PVA.

FIG. 7F is a graph showing UV-vis spectroscopy results (using a plate reader) of the solution of FIG. 7E after being frozen (i.e., in a frozen state). It can be seen that the absorbance distribution of the solution containing PVA when frozen is greater than the absorbance distribution before freezing, as illustrated by the arrows in FIGS. 7E and 7F.

FIG. 7G is a graph showing a sigmoidal dose response curve depicting a change in SPR (ΔA₅₂₀) over a change in reagent concentration (Log[A] (mg/ml)) between the initial liquid state (FIG. 7E) and the frozen state (FIG. 7F). A resulting IC50 of 0.043 and a slope n of −1.11 was then calculated.

FIG. 7H is a graph showing a sigmoidal dose response curve depicting a change in AF (ΔAF) over a change in reagent concentration (Log[A] (mg/ml)) between the initial liquid state (FIG. 7E) and the frozen state (FIG. 7F). A resulting IC50 of 0.27 and a slope n of −3.84 was then calculated.

As depicted in FIGS. 7A-7H, the calculated SPR change (ΔA₅₂₀) and AF change (ΔAF) before and after the freezing-thaw cycle were determined (standard deviation was marked with 3 times of repeats) and a fitting curve (dotted line) was plotted for each figure using the Hill equation. The calculated IC50 and n numbers tell us that, using the analysis technique described above, we can compare results for each condition (e.g., frozen, thawed) as well as each sample type with quantified metrics. The post-thaw method is easier to analyze because spectrum measurements taken during a frozen state can lead to unexpected errors (e.g., partial thawing during measurements that can skew the result) for spectroscopy. However, the frozen state can reflect the apparent distance effect between individual AuNPs during ice crystallization inhibition by analytes (here PVA), so the results obtained during a frozen state are very useful for comparing AuNPs to other materials. Here, the IC50 result for a frozen state is higher than that for a post-thaw state, which means PVA was less effective during a frozen state analysis in terms of anti-icing effects.

### 2.0 Application: Anti-Icing Effect of Chemicals and Proteins

### 2.1 Measuring Anti-Icing Activity of Chemicals and Proteins Using AuNP-Based HTS Assay

In order to confirm results, additional tests were performed. Specifically, a serial dilution assay was repeated using AuNP-TA/NTA (i.e., AuNP-probe) for different combinations of HTS, with repetitions (2 times) to confirm the results were repeatable for different combinations at different batches of AuNPs, and to confirm the results were also repeatable for the same/consistent combinations.

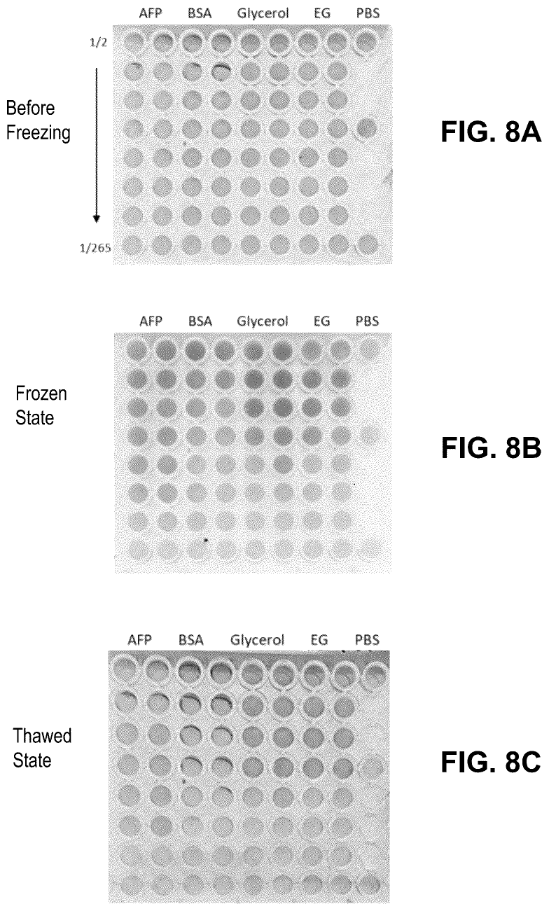

FIG. 8A is an image of a 96 well plate series filled with AuNP-TA/NTA probe solutions with different regents, serially diluted (before freezing). Specifically, the AuNP-TA/NTA probe was tested in: AFP 0.2 mg/ml (lane 1-2), BSA 0.2 mg/ml (lane 2-4), Glycerol 50% (lane 5-6), EG 50% (lane 7-8), and PBS buffer only (lane 9) as a control. The target samples were diluted by ½, ¼, ⅛, 1/16, 1/32, 1/64, 1/128, and 1/256 times (from top to bottom) by using 2×PBS and then mixed with 50 uL of 20 nM AuNP-TA/NTA probe (to make final 1×PBS and 10 nM of AuNP-TA/NTA probe in each well).

FIG. 8B is an image of the 96 well plate series of FIG. 8A after freezing (i.e., in a frozen state). Specifically, the 96 well plate series was frozen at −20° C. for 2 hours, resulting in the frozen state depicted in FIG. 5B.

FIG. 8C is an image of the 96 well plate series of FIG. 8B after thawing (i.e., in a thawed state). PBS was used as a negative control, and showed the largest color changes in contrast to the positive controls, confirming that the AuNP-TA/NTA probe was working well. Interestingly, BSA shows there are color changes depending on concentration, indicating that BSA provides low-level anti-icing effects compared to other known proteins (e.g., compared to AFP in the first two columns). This phenomenon has been discussed previously. Specifically, BSA can have a suppression effect on freezing temperatures or a small anti-icing effect in high concentrations, as any material can show a similar effect eventually in high enough concentrations. See D. E. Mitchell, T. Congdon, A. Rodger, M. I. Gibson, “Gold Nanoparticle Aggregation as a Probe of Antifreeze (Glyco) Protein-Inspired Ice Recrystallization inhibition and Identification of New IRI Active Macromolecules”, Sci. Rep. 2015, 5, 15716. This effect of BSA was also confirmed via traditional IRI test using a sucrose-based sandwich assay, but other well-known anti-icing materials showed a much higher activity, as discussed below.

Next, the anti-icing efficacy of each testing molecule was quantified using an AuNP-TA/NTA based HTS assay for screening anti-icing target materials, the change of spectrum/color, and Hill equation, in accordance with embodiments of the invention.

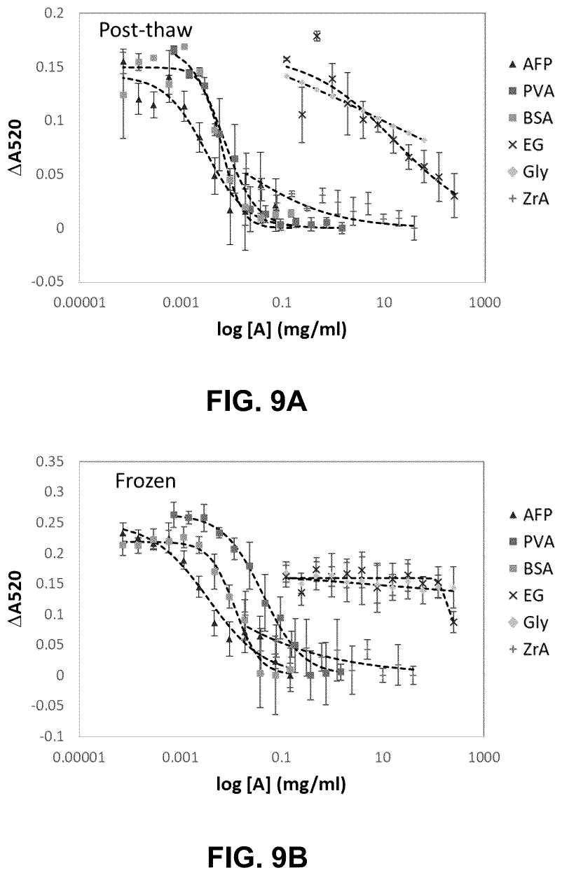

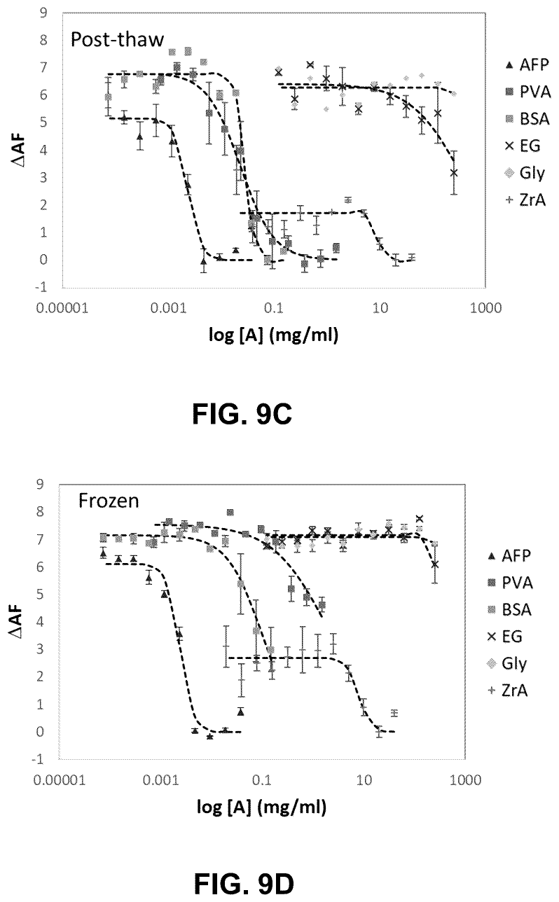

FIG. 9A is a graph showing a change in SPR (ΔA₅₂₀) over a change in reagent concentration (Log[A] (mg/ml)) for AuNP-TA/NTA probe solutions between an initial liquid phase and after being thawed from a frozen state (i.e., in a thawed state). More specifically, solutions including an AuNP-TA/NTA probe in combination with respective target materials AFP, PVA, BSA, EG, Gly and ZrA were analyzed. More specifically, target anti-icing materials tested included: AFP 0.3 mg/ml, PVA 10K 0.3%, BSA 0.3 mg/ml, EG 50%, glycerol 50%, and 80 mg/ml zirconium acetate. The buffer utilized was 1×PBS, with the exception of ZrA which was in 40 mM HCl. The target samples were diluted by ½, ¼, ⅛, 1/16, 1/32, 1/64, 1/128, 1/256, 1/512, 1/1024, 1/2048 and 1/4096 times by using 2×PBS or 80 mM HCl (for ZrA) and then mixed with 50 μL of 20 nM AuNP-TA/NTA probe (to make final 1×PBS (or 40 mM HCl for the last sample) and 10 nM of AuNP-probe in each well).

FIG. 9B is a graph showing a change in SPR (ΔA₅₂₀) over a change in reagent concentration (Log[A] (mg/ml)) for the AuNP-TA/NTA probe solutions between an initial liquid phase and a frozen state. The Hill equation was used to fit the absorbance changes at 520 nm (corresponding to the intrinsic SPR peak) for FIGS. 9A and 9B. The resulting fitting curves for AFP, PVA, BSA, EG, GLY, and ZrA are presented, with standard deviations shown based on three repeated measurements.

FIG. 9C is a graph showing a change in AF (ΔAF) over a change in reagent concentrations (Log[A] (mg/ml)) for the for the AuNP-TA/NTA probe solutions an initial liquid phase and after being thawed from a frozen state (i.e., in a thawed state). The Hill equation was applied for fitting AF changes with the changes in concentration.

FIG. 9D is a graph showing a change in AF (ΔAF) over a change in reagent concentrations (Log[A] (mg/ml)) for the AuNP-TA/NTA probe solutions between an initial liquid phase and a frozen state. The Hill equation was used to fit the AF changes (corresponding to the intrinsic SPR peak) for FIGS. 9C and 9D. The resulting fitting curves for AFP, PVA, BSA, EG, GLY, and ZrA are presented, with standard deviations shown based on three repeated measurements.

As can be seen in FIGS. 9A-9D, AFP showed the best reactivity and responsiveness even at lower concentrations. This analysis method demonstrates the very low IC50 of AFP (high anti-icing activity). High concentrations of chemical-based anti-icing materials showed very little changes in both SPR and AF for the dynamic range analyzed, which suggests the need to change the dynamic range of concentrations to further test these materials.

### 2.2. Correlation Between AuNP-Based HTS and Traditional Anti-Icing Measurements: IRI and DIS

The results of the HTS assay above were compared to a traditional IRI test and a DIS assay to find correlations. In general, individual ice crystals have a hexagonal shape to minimize the surface energy in water, while a group of ice crystals has a rounded shape. AFP that originates from fish often binds to water, resulting in the construction of prisms and/or pyramidal planes of ice lattice as the water is frozen, which results in ice having a bi-pyramidal shape (hexagonal bipyramid and hexagonal trapezohedron). In contrast, AFP that originates from insects and fungus, binds to water in a manner that creates ice with a slightly different morphology (e.g., the lemon shape), as they bind to multiple planes including the basal plane. When the temperature is lowered to a certain minus temperature, a bursting growth occurs from weak points of the ice surface not covered with AFPs (normally two tips of the bipyramid). See A. T. Rahman, T. Arai, A. Yamauchi, A. Miura, H. Kondo, Y. Ohyama, S. Tsuda, “Ice recrystallization is strongly inhibited when antifreeze proteins bind to multiple ice planes”, Sci Rep. 2019, 9(1), 2212.

Thermal hysteresis (TH) is defined as the temperature difference between the melting points and crystallization temperatures of phase-change materials (PCMs). TH can also be understood as the temperature range in which ice neither melts nor grows. TH can be expressed by the following equation EQ(5), wherein Tₘ is the melting temperature of a reagent and T_{f} is the freezing temperature of a reagent:

$$TH = \vert {T}_{m} - {T}_{f} \vert . \qquad EQ ( 5 )$$

A TH value represents the strength of ice-growth inhibition of an anti-icing material. Also, if there is an anti-icing effect, ice recrystallization inhibition, IRI, can often be seen during a rewarming process of a frozen sample with anti-icing molecules therein. To test IRI effects, often high concentrations of sucrose are used to keep the ice crystals separate, without fusion, so that a change in the size of ice crystals over time can be observed with a change in temperature. A DIS assay may be used to focus on the specific ice growth shape depending on which facet or side of ice (basal plane, pyramidal plane and prism plane) has an interaction with the molecules of interest. Both an IRI test and a DIS test were performed using a sandwich-type assay to observe the correlations between results of the AuNP-based HTS assay of the present invention and results of the IRI and DIS tests in terms of anti-icing effects.

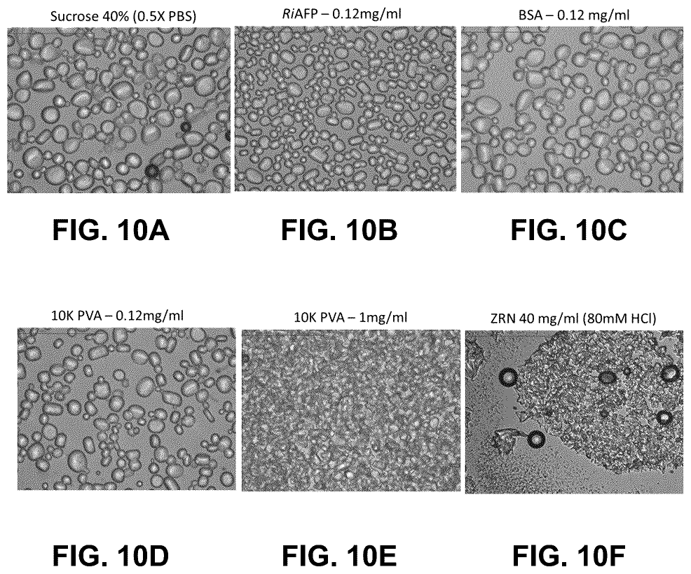

FIGS. 10A-10F show images of IRI assay results for a plurality of test materials in buffers, for the purpose of comparison. More specifically, FIGS. 10A-10F show respective anti-icing samples tested including: sucrose 40% (as a negative control), AFP (RiAFP) 0.12 mg/ml, BSA 0.12 mg/ml, 10K PVA 0.12 mg/ml, 10K PVA 1 mg/ml, and zirconium acetate (ZrN) 40 mg/ml. The buffer utilized was 0.5×PBS, with the exception of ZrN which was in 80 mM HCl. For the IRI assay, each sample was prepared with 40% sucrose as a final concentration. The following procedures were performed for each sample. Initially, 2 μL of sample was dropped between two round coverslips (borosilicate, d=1.3˜1.5 cm) and sealed with grease. The sample was then inserted into a Linkam® Scientific cryostage with liquid nitrogen (LN2) control. The sample was first frozen at −50° C. for 1 min, then the temperature was increased to −20° C. at a rate of 10° C./min, followed by a slow increase in temperature to −10° C. at a speed of 1° C./min. A sample image was then taken after annealed at −10° C. for 30 minutes without interruption.

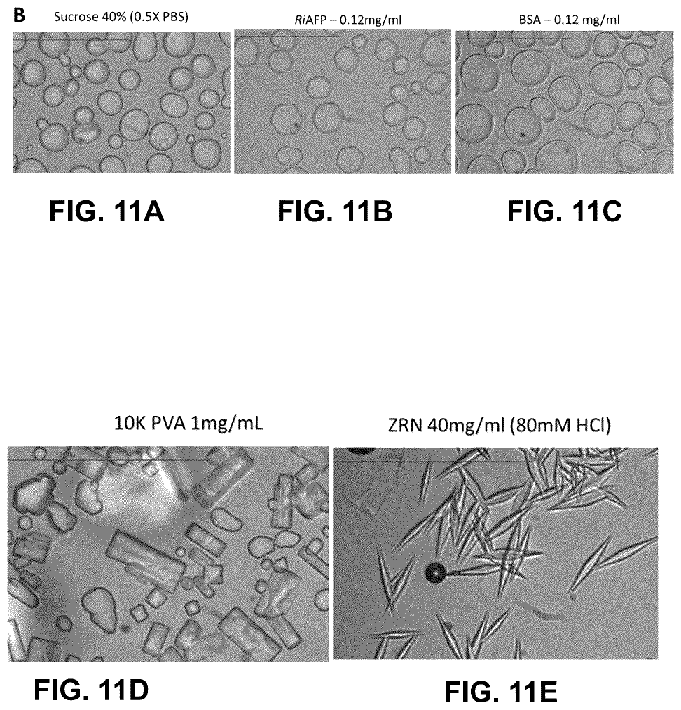

FIGS. 11A-11E show images of DIS assay results for a plurality of test materials in buffers, for the purpose of comparison. More specifically, FIGS. 11A-11E show respective anti-icing samples tested including: sucrose 40% (as a negative control), AFP (RiAFP) 0.12 mg/ml, BSA 0.12 mg/ml, 10K PVA 1 mg/ml, and ZrN 40 mg/ml. All samples have sucrose 40% as a co-solute and 0.5×PBS as a buffer, with the exception of ZrN which was in 80 mM HCl. For the DIS assay, the sample preparation was the same as in the IRI assay described above, but the temperature was controlled at a much slower rate (0.1˜0.2° C./min) to control the temperature between melting and freezing ice before taking an image. Each sample was in a near TH zone with a temperature control of 0.1˜0.2° C./min. To compare IRI results, the ice size was measured and compared to that of sucrose 40% only. The ice crystal size during recrystallization process in −10° C. and literature searched TH were well correlated with the IC50 result measured by the above-identified AuNP-based HTS assay.

### 3.0 AuNP-Based HTS Assay Methods and Features

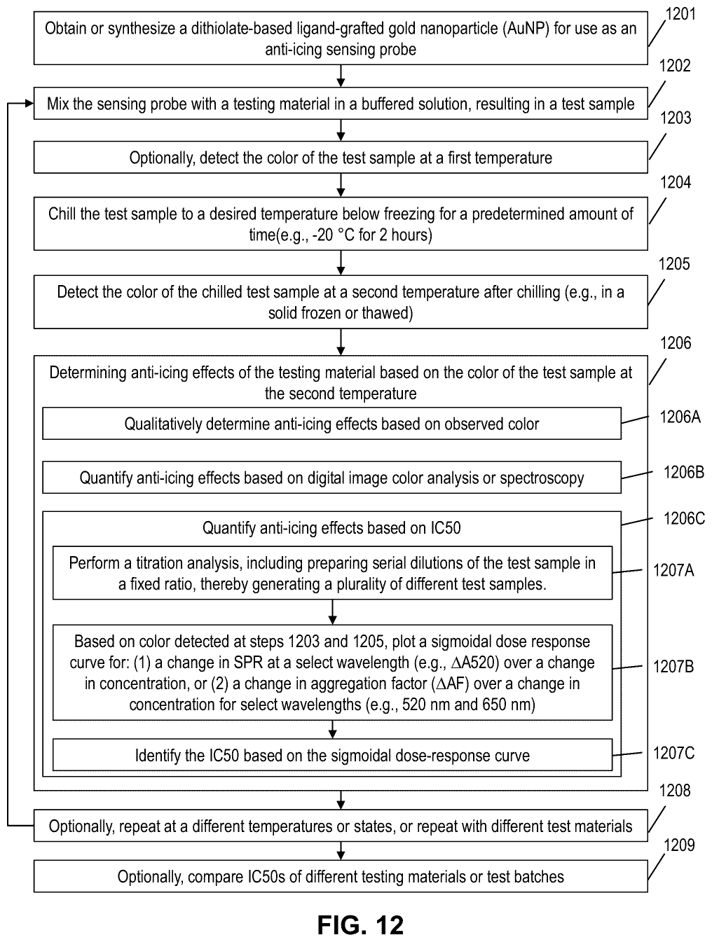

FIG. 12 shows a flowchart of an exemplary AuNP-based HTS assay method in accordance with aspects of the present invention. Implementations of the invention may be used to generate qualitative anti-icing information. Other implementations of the invention may be used to generate quantitative anti-icing information. In some embodiments, an IC50 number is generated for a particular testing material that indicates its anti-icing effect, which can be compared to other testing materials (e.g., different proteins) using the same analysis method (e.g., same NP size and buffer). Qualitative and quantitative method steps are discussed in more detail below.

In implementations, at 1201, a dithiolate-based ligand-grafted gold nanoparticle (AuNP) is produced or obtained for use as an AuNP-based colorimetric sensing probe (hereafter AuNP-probe). In implementations, the dithiolate-based ligand has a bidentate binding group, such as thioctic acid (TA) with disulfide and its reduced form of dihydrolipoic acid (DHLA) with dithiol. The reduced form may be made by methods known in the art. In embodiments, the dithiolate-based ligand is modified with one or more terminal functional groups with an additional ethylene glycol moiety between binding groups and terminal functional groups to enhance solubility in different buffers or at different pH. For example, types of terminal functional groups that may be utilized include: a carboxylic acid (COOH) group, a hydroxyl (OH) group, a methoxy (OCH₃) group, a sulfonate (SO₃⁻) group, an amine (NH₂) group, an azide (N₃) group, and a nitrilotriacetic acid (NTA) group. Advantageously dithiolate-based ligands of the present disclosure have a much stronger binding to a surface of a nanoparticle than a single thiol (e.g., mercaptosuccinic acid (MSA)). Exemplary dithiolate-based ligands for use with embodiments of the invention include TA, TA-NTA, TA-OEG₂ and TA-SO₃⁻, depicted in FIGS. 4A-4D. Methods for synthesizing these ligands are discussed below. In implementation, the dithiolate-based ligand-grafted AuNP is made utilizing the same method as the Au-TA/NTA discussed above.

In embodiments, at 1202, an analyte to-be-tested for anti-icing activity (hereafter “testing material”) is mixed with the AuNP-probe in a buffered solution, resulting in a test sample. The testing material may be an organic chemical, inorganic chemical or biomolecule, for example. The buffer may be selected based on the testing material. For example, the buffer may be: PBS, Tris/BorateEDTA, or HCl, or acetate buffer. Step 1202 may include the substep of diluting the test sample in a fixed ratio to generate different test samples.

Optionally, at 1203, a color (first color) of the test sample(s) at a first temperature (e.g., room temperature) is detected, wherein the test sample(s) is in a first state or phase (e.g., liquid phase). Detection of the color may comprise capturing a color image of the test sample using UV-vis spectroscopy or a plate reader (Spark® microplate reader by Tecan® Trading Ag). Detecting the color of a test sample(s) at 1203 (e.g., via spectroscopy or color image) is required for determining an IC50 of a testing material. Step 1203 may include the substep of changing the temperature of the test sample(s) to the first temperature.

At 1204, the test sample(s) is chilled at a desired temperature below freezing for a predetermined amount of time, resulting in a test sample(s) at a second temperature (below freezing), wherein the test sample(s) may be in a second state or phase (e.g., frozen or vitrified). In one example, the test sample(s) is chilled for 2 hours at −20° C.

In embodiments, at 1205, a color (second color) of the test sample(s) is detected subsequent to step 1204. In one example, the color is detected while the test sample(s) is still chilled (e.g., in a frozen state). In another example, the test sample(s) is allowed to warm up or thaw to a third temperature (e.g., room temperature) before the color is detected (e.g., in a thawed state). In one example, the test sample is in a first, liquid state, at the first temperature and is in a second, frozen state, at the second temperature. In another example, the test sample is in a first, liquid state, at the first temperature, and is in a second, vitrified state, at the second temperature. In yet another example, the test sample is in a first, liquid state, at the first temperature, and is in a second, thawed state, at a third temperature. Detection of the color may comprise capturing a color image of the test sample as described above. Alternatively, detecting the color of a test sample(s) may comprise a qualitative visual observation by a user. In general, the intensity of a red color of a test sample when frozen or thawed correlates with the anti-icing effects of the test material. In implementations, a HTS analysis of multiple testing materials may be performed using only a visual analysis of test samples, such as when screening a number of testing materials for anti-icing effects.

In implementations, at 1206, the anti-icing effect of a testing material is determined based on the color of the test sample(s) detected at step 1205. In general, the intensity of a color of the test sample correlates with the degree to which the testing material binds with the AuNP-probe, which in turn indicates the anti-icing effect of the testing material. Thus, the anti-icing effect of a testing material can be determined qualitatively or quantitatively in accordance with embodiments of the invention.

In implementations, at substep 1206A, a user qualitatively determines the anti-icing effect of a testing material by comparing a color of a test sample(s) with a negative control sample, a color chart showing colors indicative of different anti-icing effects, or other test samples.

In other implementations, at substep 1206B, a user quantifies the anti-icing effect of a testing material based on digital image color analysis or spectroscopy. For example, the color of a negative control sample may be compared to a color of a test sample using digital image colorimetry (DIC).

In yet other implementations, at substep 1206C, a user quantifies the anti-icing effect of a testing material based on a calculated IC50 number for the testing material. The following steps may be utilized to calculate the IC50 of a testing material.

At step 1207A, a titration analysis is performed, including preparing serial dilutions of the test sample in a fixed ratio, thereby generating a plurality of different test samples. This step may be incorporated into step 1202.

At step 1207B, based on the color detected at steps 1203 and 1205, a sigmoidal dose response curve is plotted for one or both of: (1) a change in SPR at a select wavelengths (e.g., 4520) over a change in concentration, and (2) a change in aggregation factor (ΔAF) over a change in concentration for select wavelengths (e.g., 520 nm and 650 nm). In implementations, the Hill Equation is utilized to generate the sigmoidal dose response curve(s). However, other methods of generating the dose-response curve may be utilized.

At step 1207C, an IC50 number is determined based on a sigmoidal dose-response curve of step 1207B. In implementations, a first IC50 number may be determined based on SPR, and another IC50 number may be determined based on AF. In general, the lower the IC50, the better the anti-icing effects of the test material. When relying on an AF dose response curve, a user must select an AF by choosing a ratio of a first absorbance at a first wavelength to a second absorbance at a second wavelength. In one example, the AF comprises a first absorbance at 520 nm and the second absorbance at 650 nm. When relying on an SPR dose response curve, a user must select a wavelength at which to measure a change in SPR. In one example, the wavelength comprises 530 nm. Step 1207C may also include the determination of a slope n of the sigmoidal dose-response curve.

Optionally, at step 1208, steps 1202-1206 may be repeated for different temperatures or states, or may be repeated with different testing materials. For example, a user may wish to perform an analysis method including steps 1202-1206 to obtain the ID50 of a test material using frozen test samples, and may then wish to perform the same steps to obtain the ID50 of a test material using thawed test samples.

Optionally, a user may compare IC50s of different testing materials, or of different test batches having the same analysis methodology. For example, the IC50 of two different testing materials may be compared wherein both IC50s are derived using an SPR sigmoidal dose response curve for a wavelength of 520 nm. In another example, the IC50s of the same testing material derived from two different test batches may be compared, where both batches derived the IC50 from an AF sigmoidal dose-response curve with an absorbance range of 520 nm and 650 nm.

Implementations of the above-identified method may be accomplished utilizing a colorimetric assay kit. In embodiments, a colorimetric assay kit for determining anti-icing effects of a test material is provided, including a dithiolate-based ligand-grafted gold nanoparticle probe (AuNP-probe) for mixing with respective test samples of testing materials in solution, wherein changes of color of the test sample with changes in temperature/state over time indicates anti-icing effects of the testing materials. The kit may also include a plurality of sample wells configured to house the respective test samples. The sample wells may be in the form of a standard sample well plate series configured to be undamaged by repeated freezing and thawing cycles. In embodiments, the colorimetric assay kit includes a buffer solution for diluting the test material in the test samples. Further, implementations of the colorimetric assay kit include instructions for determining anti-icing effects of the test material using the colorimetric assay kit and a provided color chart showing a plurality of different colors for associated testing materials and molecular states (e.g., frozen, and/or thawed states). In embodiments, a user of the colorimetric assay kit may qualitatively determine anti-icing effects of a testing material by comparing a color of a test sample with the colors of test samples in the color chart, wherein the test samples in the color chart are associated with anti-icing effects or IC50 numbers.

As noted above, a variety of dithiolate-based ligands may be grafted to gold nanoparticle to create an AuNP-probe in accordance with embodiments of the invention. It has been shown that TA-based bidentate ligands can be used as versatile ligands for various types of NPs (e.g., AuNP, AgNP, QD). NPs modified with TA-based bidentate ligands have showed excellent colloidal stability compared to monothiol-based ligands or other types of ligands, and despite high concentrations of ions (2 M NaCl and pH 2-13) or aggregation-causing dithiothreitol (DTT), such NPs did not show optical and morphological transformation for several months. Owing to this durability of TA-based ligands, implementations of the invention provide a more stable AuNP-based colorimetric sensing probe that is applicable for HTS of the anti-icing activity of target materials with high efficiency and versatility for different kinds of target materials in different solvent/buffer or environmental conditions.

### 4. TA-OEG₂ Synthesis

Initially, it is noted that ligand TA depicted in FIG. 4A is commercially available. Additionally, synthesis methods for ligand TA-NTA in FIG. 4B, and TA-SO₃⁻; in FIG. 4D are available in the literature, and are not discussed herein. Ligand TA-OEG₂ of FIG. 4C may be synthesized utilizing the following steps and precursors.

### Precursor I: N,N-Bis{2-[2-(2-hydroxyethoxy)ethoxy]ethoxy}-N′-(tert-butoxycarbonyl)ethylenediamine

A mixture of 2-[2-(2-Chloroethoxy)ethoxy]ethanol (20.4 g, 0.121 mol), sodium iodide (NaI) (30.0 g, 0.200 mol), sodium carbonate (Na₂CO₃) (90.0 g, 0.849 mol) and acetonitrile (CH₃CN) (500 mL) was refluxed for 7.5 hours (h) under nitrogen gas (N₂), and N-(tert-butoxycarbonyl)ethylenediamine (8.30 mL, 5.24×10⁻² mol) was added. The reaction mixture was further refluxed for 64 h under N₂. After cooling, the white solid was filtered off and washed with CH₃CN. The filtrate was evaporated and deionized water (DI H₂O) (150 mL) was added to the residue. The aqueous layer was washed twice with ether, and the product was extracted with chloroform (CHCl₃) (4 times). The combined CHCl₃ layers were dried over sodium sulfate (Na₂SO₄). The inorganic salt was filtered off and the solvent was evaporated to yield the product as oil. Yield=20.67 grams (g) (93% based on 8.30 milliliters (mL) of N-(tert-butoxycarbonyl)ethylenediamine). ¹H NMR (400 MHZ, CDCl₃): δ 5.71 (br s, 1H, NH), 3.73 (t, 4H, J=5.8 Hz, OCH₂), 3.58-3.70 (m, 12H, OCH₂), 3.55 (t, 4H, J=7.4 Hz, OCH₂), 3.17 (m, 2H, NHCH₂), 2.73 (t, 4H, J=7.2 Hz, NCH₂), 2.62 (t, 2H, J=7.6 Hz, NCH₂), 1.45 (s, 9H, t-Bu).

### Precursor II: N,N-Bis{2-[2-(2-hydroxyethoxy)ethoxy]ethoxy}ethylenediamine

A mixture of Precursor I (8.145 g, 1.92×10⁻² mol) and 4M hydrogen chloride (HCl) in dioxane (25 mL) was stirred at room temperature for 1 h under N₂. The solvent was evaporated. DI H₂O (80 mL) was added to the residue, and the aqueous layer was washed with ether (2 times), basified with sodium hydroxide (NaOH) (pH>13), and saturated with NaCl. The product was extracted several times with CHCl₃. The combined organic layers were dried over Na₂SO₄. The inorganic salt was filtered off and the filtrate was evaporated to obtain the product as oil. Yield=5.817 g (93% based on 8.145 g of I). ¹H NMR (400 MHZ, CDCl₃): δ 3.75 (t, 4H, J=5.8 Hz, OCH₂), 3.64 (m, 8H, OCH₂), 3.60 (t, 4H, J=5.8 Hz, OCH₂), 3.52 (t, 4H, J=6.8 Hz, OCH₂), 2.84 (t, 2H, J=7.4 Hz, NCH₂), 2.72 (t, 4H, J=6.8 Hz, NCH₂), 2.66 (t, 2H, J=7.4 Hz, NCH₂).

### Final Ligand: TA-(OEG)₂

A mixture of thioctic acid (0.600 g, 2.91×10⁻³ mol) and N-Ethoxycarbonyl-2-ethoxy-1,2-dihydroquinoline (EEDQ) (0.887 g, 4.04×10⁻³ mol) in dichloromethane (CH₂Cl₂) (6.0 mL) was stirred at room temperature for 20 minutes under N₂. To the reaction mixture was added Precursor II (1.002 g, 3.09×10⁻³ mol) in CH₂Cl₂ (4.0 mL). The reaction was monitored by thin-layer chromatography (TLC), and EEDQ (0.203 g, 8.21×10⁻⁴ mol) was further added after 8.5 h. The reaction mixture was stirred overnight under N₂. The solvent was evaporated, and the residue was chromatographed on silica gel with CHCl₃:methanol (MeOH) (10:1) as an eluent to purify the product. Yield=1.051 g (70% based on 0.600 g of thioctic acid). ¹H NMR (400 MHz, CDCl₃): δ 7.22 (br s, 1H, NH), 3.72 (t, 4H, J=5.8 Hz, OCH₂), 3.5-3.7 (m, 17H, OCH₂ and CH), 3.32 (q, 2H, J=7.2 Hz, NHCH₂), 3.06-3.23 (m, 2H), 2.71 (t, 4H, J=6.9 Hz, NCH₂), 2.61 (t, 2H, J=7.2 Hz, NCH₂), 2.40-2.52 (m, 1H), 2.22 (t, 2H, J=9.9 Hz, CH₂CO), 1.84-1.97 (m, 1H), 1.58-1.78 (m, 4H), 1.37-1.55 (m, 2H).

Based on the above, it can be understood that implementations of the dithiolate-grafted AuNP probe of the present invention provide: (1) improved stability and buffer resilience essential for the HTS to screen various types of targets, (2) improved repeatability of assays of the present invention, and enhance dynamic range; (3) for the use of AuNP as a “molecular ruler” to reflect the apparent distance between AuNPs and ice size, (4) for measuring anti-icing effects at different phases of a sample (e.g., liquid, solid, mixed, supercooled, frozen, vitrified), various temperatures (e.g., room temperature to frozen), different freezing times (e.g. 1 hours to days), and multiple freezing-thaw cycles over months to understand time-dependent activity during freezing and warming of samples; (5) for a fast absorbance measurement and a fast color image measurement suitable for HTS; and (6) for new analysis protocols using a titration method or reagents and IC50 calculations of an HTS assay, which deliver quantified and standardized values of anti-icing efficacy. Applicant applied HTS using dithiolate-grated AuNP probe to measure different anti-icing targets materials, including organic chemicals (e.g., poly(vinyl alcohol), ethylene glycol, sucrose, TA-based ligands), inorganic chemicals (e.g., zirconium acetate (Zr(OAc)₄)) and biomolecules (e.g., AFP, bovine serum albumin) and found that results correlate with traditional time-consuming analysis methods (e.g., sandwich assay for IRI activity).

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A compound having the formula:

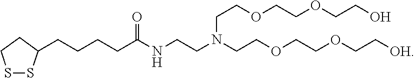 [SMILES: O=C(CCCCC1CCSS1)NCCN(CCOCCOCCO)CCOCCOCCO]

2. A compound having the formula:

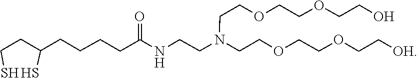 [SMILES: O=C(CCCCC(S)CCS)NCCN(CCOCCOCCO)CCOCCOCCO]

3. A method comprising:
reacting 2-[2-(2-chloroethoxy)ethoxy]ethanol with N-(tert-butoxycarbonyl)ethylenediamine to form N,N-bis{2-[2-(2-hydroxyethoxy)ethoxy]ethoxy}-N′-(tert-butoxycarbonyl)ethylenediamine;
treating the N,N-bis{2-[2-(2-hydroxyethoxy)ethoxy]ethoxy}-N′-(tert-butoxycarbonyl)ethylenediamine with acid to form N,N-bis{2-[2-(2-hydroxyethoxy)ethoxy]ethoxy}ethylenediamine;
reacting the N,N-bis{2-[2-(2-hydroxyethoxy)ethoxy]ethoxy}ethylenediamine with thioctic acid to form the product:

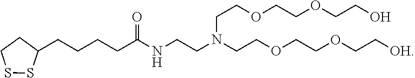 [SMILES: O=C(CCCCC1CCSS1)NCCN(CCOCCOCCO)CCOCCOCCO]

4. The method of claim 3, further comprising:
reducing the product of claim 3 to form the compound: 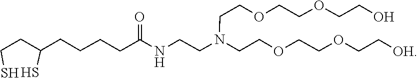 [SMILES: O=C(CCCCC(S)CCS)NCCN(CCOCCOCCO)CCOCCOCCO]
